(19) United States
(12) Patent Application Publication
(10) Pub. No.: US 2006/0204452 A1
(43) Pub. Date: Sep. 14, 2006

(54) Antimicrobial film-forming dental compositions and methods

(76) Inventors: Bhaskar V. Velamakanni, Woodbury, MN (US); Surnita B. Mitra, West St. Paul, MN (US); Danli Wang, Shoreview, MN (US); Matthew T. Scholz, Woodbury, MN (US); Paul A. Burgio, Grant, MN (US); Mahfuza B. Ali, Mendota Heights, MN (US)

Correspondence Address: 3M INNOVATIVE PROPERTIES COMPANY, PO BOX 33427, ST. PAUL, MN, 55133-3427, US

(21) Appl. No.: 11/077,658

(22) Filed: Mar. 10, 2005

(51) Int. Cl. A61K 8/81; A61K 8/36

(52) U.S. Cl. 424/49

(57) ABSTRACT

The present application provides dental compositions, methods of making, and methods of using dental compositions that include an antimicrobial lipid component and a water-dispersible, polymeric film-former.

### BACKGROUND

Due to an alarming increase in drug-resistant bacterial infections, antibiotic use in oral care has been limited for the management of active infectious diseases. Typically, antiseptics and disinfectants have been used in the oral environment in the war against disease-causing microorganisms. For example, glutaraldehyde, chlorhexidine, quaternary ammonium salts, triclosan, etc., are often used for oral hygiene in oral rinses, dentrifices, and dental restorative materials such as etchants, varnishes, adhesives, etc. Recently, reactive polymers of quaternary ammonium salts are being used as immobilized antimicrobial dental adhesives.

Such antimicrobial materials often have limited effectiveness against a narrow spectrum of pathogenic bacteria. For example, cationic quaternary ammonium salts tend to chelate with metal ions in the oral cavity and lose their effectiveness. Thus, new dental compositions having antimicrobial activity are needed.

### SUMMARY OF THE INVENTION

The present invention provides dental compositions having antimicrobial activity that are useful for local/topical treatment (therapeutic or prophylactic) of conditions that are caused, or aggravated by, microorganisms. More specifically, dental compositions of the present invention are useful for preparing dental materials and articles that are effective against one or more microbes (including viruses, bacteria, yeast, mold, fungi, micoplasma, and protozoa), particularly in the oral environment.

In one embodiment, the present invention provides a dental composition that includes: an effective amount of an antimicrobial lipid component including a (C7-C12)saturated fatty acid ester of a polyhydric alcohol, a (C8-C22)unsaturated fatty acid ester of a polyhydric alcohol, a (C7-C12)saturated fatty ether of a polyhydric alcohol, a (C8-C22)unsaturated fatty ether of a polyhydric alcohol, an alkoxylated derivative thereof, or combinations thereof, wherein the alkoxylated derivative has less than 5 moles of alkoxide per mole of polyhydric alcohol; with the proviso that for polyhydric alcohols other than sucrose, the esters comprise monoesters and the ethers comprise monoethers, and for sucrose the esters comprise monoesters, diesters, or combinations thereof, and the ethers comprise monoethers, diethers, or combinations thereof; and a water-dispersible, polymeric film-former.

For certain embodiments, the water-dispersible, polymeric film-former includes a repeating unit that includes a polar or polarizable group. For certain embodiments, the polar or polarizable group is derived from vinylic monomers. For certain embodiments, the water-dispersible polymeric film-former further includes a repeating unit that includes a fluoride releasing group, a repeating unit that includes a hydrophobic hydrocarbon group, a repeating unit that includes a graft polysiloxane chain, a repeating unit that includes a hydrophobic fluorine-containing group, a repeating unit that includes a modulating group, or combinations thereof.

For certain embodiments, the water-dispersible polymeric film-former includes reactive groups. For certain embodiments, the reactive groups are selected from the group consisting of ethylenically unsaturated groups, epoxy groups, silane moieties capable of undergoing a condensation reaction, and combinations thereof.

For certain embodiments, the water-dispersible polymeric film-former includes a polymer having repeating amide functional groups. For certain embodiments, the water-dispersible polymeric film-former includes a polymer having repeating acrylate functional groups. For certain embodiments, the water-dispersible polymeric film-former includes a polymer having repeating N-isopropylamide functional groups. For certain embodiments, the water-dispersible polymeric film-former includes a polymer having repeating urethane functional groups.

For certain embodiments, the antimicrobial lipid component includes glycerol monolaurate, glycerol monocaprate, glycerol monocaprylate, propylene glycol monolaurate, propylene glycol monocaprate, propylene glycol monocaprylate, or combinations thereof.

For certain embodiments, the antimicrobial lipid component is present in an amount of at least 0.1 wt-%.

For certain embodiments, the antimicrobial lipid component includes a monoester of a polyhydric alcohol, a monoether of a polyhydric alcohol, or an alkoxylated derivative thereof, and the antimicrobial lipid component further includes no greater than 15 wt-%, based on the total weight of the antimicrobial lipid component, of a di- or tri-ester, a di- or tri-ether, alkoxylated derivative thereof, or combinations thereof.

For certain embodiments, dental compositions of the present invention can further include an effective amount of an enhancer component distinct from the antimicrobial lipid component. For certain embodiments, the enhancer component can include a carboxylic acid. For certain embodiments, the enhancer component can include an alpha-hydroxy acid. For certain embodiments, the enhancer component includes an alpha-hydroxy acid, a beta-hydroxy acid, a chelating agent, a (C1-C4)alkyl carboxylic acid, a (C6-C12)aryl carboxylic acid, a (C6-C12)aralkyl carboxylic acid, a (C6-C12)alkaryl carboxylic acid, a phenolic compound, a (C1-C10)alkyl alcohol, an ether glycol, or combinations thereof. For certain embodiments, the total concentration of the enhancer component relative to the total concentration of lipid component is within a range of 10:1 to 1:300, on a weight basis.

For certain embodiments, dental compositions of the present invention can further include an effective amount of a surfactant component distinct from the antimicrobial lipid component. For certain embodiments, the surfactant component can include a sulfonate surfactant, a sulfate surfactant, a phosphonate surfactant, a phosphate surfactant, a poloxamer surfactant, a cationic surfactant, or mixtures thereof. For certain embodiments, the surfactant component can include a sulfonate surfactant, a sulfate surfactant, a poloxamer surfactant, or mixtures thereof. For certain embodiments, the surfactant component is dioctyl sodium sulfosuccinate. For certain embodiments, the surfactant component is a poloxamer including a copolymer of polyethylene oxide and polypropylene oxide. For certain embodiments, the total concentration of the surfactant component to the total concentration of antimicrobial lipid component is within a range of 5:1 to 1:100, on a weight basis.

For certain embodiments, dental compositions of the present invention can further include a hardenable component. For certain embodiments, the hardenable component can include an ethylenically unsaturated compound. For certain embodiments, the ethylenically unsaturated compound is a (meth)acrylate compound. For certain embodiments, the ethylenically unsaturated compound is selected from the group consisting of an ethylenically unsaturated compound with acid functionality, an ethylenically unsaturated compound without acid functionality, and combinations thereof. For certain embodiments, dental compositions that include a hardenable component can further include an initiator system.

For certain embodiments, dental compositions of the present invention can further include a filler, a solvent, a thermally responsive viscosity modifier, or combinations thereof.

The present invention provides methods of forming a polymeric film coating layer on an oral cavity surface. In one embodiment, the method includes: combining an antimicrobial lipid component and a water-dispersible, polymeric film-former to form a dental composition of the present invention; applying the composition to the oral cavity surface; and allowing the film coating to form on the oral cavity surface.

In another embodiment, the method includes: combining an antimicrobial lipid component, a water-dispersible, polymeric film-former, and a volatile solvent to form a dental composition of the present invention; applying the composition to the oral cavity surface; and allowing the evaporation of at least a portion of the volatile solvent to form the film coating layer on the oral cavity surface.

In such methods, the oral cavity surface can be dentine and/or enamel.

The methods can involve the use of a dental composition that includes a hardenable component. In such embodiments, the methods include a step of hardening the composition after the applying step.

The film coating layer can be continuous or discontinuous. The film coating layer can be porous or non-porous.

### DEFINITIONS

As used herein, a “hardenable” component refers to one that is capable of polymerization and/or crosslinking reactions including, for example, photopolymerization reactions and chemical polymerization techniques (e.g., ionic reactions or chemical reactions forming radicals effective to polymerize ethylenically unsaturated compounds, oxirane compounds, etc.) involving one or more compounds capable of hardening. Hardening reactions also include acid-base setting reactions such as those common for cement forming compositions (e.g., zinc polycarboxylate cements, glass-ionomer cements, etc.).

As used herein, “water-dispersible, polymeric film-former” refers to a polymer that can be dispersed or dissolved in a solvent or cosolvent (optionally including water) to afford a dispersion (e.g., an emulsion, stable liquid dispersion, paste, gel, cream, and the like) or solution, such that when the dispersion or solution is coated on a hard surface (e.g., tooth enamel or dentine) and the solvent or cosolvent evaporated, there remains on the hard surface a film coating that is generally resistant to wash off by water, saliva, and/or hot liquids. The “water-dispersible polymeric film-former” typically has a water solubility of less than 1.0% by weight, preferably less than 0.1% by weight, and more preferably less than 0.01% by weight; and has a weight average molecular weight (MW) of typically greater than 500, preferably greater than 1000, and more preferably greater than 5000; the MW typically is no greater than 100,000.

As used herein, a “dental composition” refers to film-forming compositions used in the oral environment including, for example, dental gels. These compositions can be used, for example, as coatings, varnishes, sealants, primers, cavity cleansing agents, desensitizers, cavity liners, colorants, whiteners, remineralizing agents, drug delivery, agents, and combinations thereof.

As used herein, a “thermally responsive composition” refers to a composition that includes water and a thermally responsive viscosity modifier, and that can be applied in a low viscosity state at room temperature to an oral cavity surface (e.g., to a tooth surface) upon which the composition increases in viscosity to a highly viscous state (e.g. to a gel-like composition).

As used herein, “(meth)acryl” is a shorthand term referring to “acryl” and/or “methacryl.” For example, a “(meth)acryloxy” group is a shorthand term referring to either an acryloxy group (i.e., CH₂═CHC(O)O—) and/or a methacryloxy group (i.e., CH₂═C(CH₃)C(O)O—).

“Effective amount” means the amount of the antimicrobial lipid component plus the enhancer component (when present in a composition) and/or the surfactant component (when present in a composition), as a whole, provides an antimicrobial (including, for example, antiviral, antibacterial, or antifungal) activity that reduces, prevents, or eliminates one or more species of microbes such that an acceptable level of the microbe results. Typically, this is a level low enough not to cause clinical symptoms, and is desirably a non-detectable level. It should be understood that in the compositions of the present invention, the concentrations or amounts of the components, when considered separately, may not kill to an acceptable level, or may not kill as broad a spectrum of undesired microorganisms, or may not kill as fast; however, when used together such components provide an enhanced (preferably synergistic) antimicrobial activity (as compared to the same components used alone under the same conditions).

“Enhancer” means a component that enhances the effectiveness of the antimicrobial lipid component such that when the composition less the antimicrobial lipid component and the composition less the enhancer component are used separately, they do not provide the same level of antimicrobial activity as the composition as a whole. For example, an enhancer component in the absence of the antimicrobial lipid component may not provide any appreciable antimicrobial activity. The enhancing effect can be with respect to the level of kill, the speed of kill, and/or the spectrum of microorganisms killed, and may not be seen for all microorganisms. In fact, an enhanced level of kill is most often seen in Gram negative bacteria such as Escherichia coli. An enhancer may be a synergist such that when combined with the remainder of the composition, the composition as a whole displays an activity that is greater than the sum of the activity of the composition less the enhancer component and the composition less the antimicrobial lipid component.

“Microorganism” or “microbe” or “microorganism” refers to bacteria, yeast, mold, fungi, protozoa, mycoplasma, as well as viruses (including lipid enveloped RNA and DNA viruses).

“Antiseptic” means a chemical agent that kills pathogenic and non-pathogenic microorganisms. Preferred antiseptics exhibit at least a 4 log reduction of both P. aeruginosa and S. aureus in 60 minutes from an initial inoculum of 1-3×10⁷ cfu/ml when tested in Mueller Hinton broth at 35° C. at a concentration of 0.25 wt-% in a Rate of Kill assay using an appropriate neutralizer as described in “The Antimicrobial Activity in vitro of chlorhexidine, a mixture of isothiazolinones (Kathon C G) and cetyl trimethyl ammonium bromide (CTAB),” G. Nicoletti et al., Journal of Hospital Infection, 23, 87-111 (1993). Antiseptics generally interfere with the cellular metabolism and/or the cell envelope. Antiseptics are sometimes referred to as disinfectants, especially when used to treat hard surfaces.

“Antimicrobial lipid” means an antiseptic having at least one (C6)alkyl or alkylene chain (preferably at least one (C7) chain and more preferably at least one (C8) chain), and preferably having a solubility in water of no greater than 1.0 gram per 100 grams (1.0 g/100 g) deionized water. Preferred antimicrobial lipids have a solubility in water of no greater than 0.5 g/100 g deionized water, more preferably, no greater than 0.25 g/100 g deionized water, and even more preferably, no greater than 0.10 g/100 g deionized water. Solubilities are determined using radiolabeled compounds as described under “Conventional Solubility Estimations” in Solubility of Long-Chain Fatty Acids in Phosphate Buffer at pH 7.4, Henrik Vorum et al., in Biochimica et. Biophysica Acta., 1126, 135-142 (1992). Preferred antimicrobial lipids have a solubility in deionized water of at least 100 micrograms (μg) per 100 grams deionized water, more preferably, at least 500 μg/100 g deionized water, and even more preferably, at least 1000 μg/100 g deionized water. The antimicrobial lipids preferably have a hydrophile/lipophile balance (HLB) of at most 6.2, more preferably at most 5.8, and even more preferably at most 5.5. The antimicrobial lipids preferably have an HLB of at least 3, preferably at least 3.2, and even more preferably at least 3.4.

“Fatty” as used herein refers to a straight or branched chain alkyl or alkylene moiety having at least 6 (odd or even number) carbon atoms, unless otherwise specified.

The term “comprises” and variations thereof do not have a limiting meaning where these terms appear in the description and claims.

As used herein, “a,” “an,” “the,” “at least one,” and “one or more” are used interchangeably. The term “and/or” means one or all of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### DETAILED DESCRIPTION OF ILLUSTRATIVE EMBODIMENTS

The present invention provides dental compositions that include an antimicrobial lipid component. Methods of making and using such dental compositions are also provided. Such compositions have antimicrobial activity and are useful for local/topical treatment (therapeutic or prophylactic) of conditions that are caused, or aggravated by, microorganisms. More specifically, such compositions are useful for preparing dental materials and articles that are effective against one or more microbes (including viruses, bacteria, yeast, mold, fungi, micoplasma, and protozoa), particularly in the oral environment.

The present invention provides dental compositions that include an antimicrobial lipid component and a polymeric film-former, and optionally a hardenable component. Such dental compositions are typically prepared by combining the antimicrobial lipid component with a water-dispersible, polymeric film-former and optionally a hardenable component. Other optional components of the dental compositions of the present invention include enhancers, surfactants, and fillers, for example.

The dental compositions of the present invention have antimicrobial activity and preferably are active against a broad spectrum of bacteria including Gram-positive and Gram-negative bacteria. Certain preferred embodiments have good to excellent activity against Streptococcus mutans (S. mutans) bacteria. S. mutans has the tendency to adhere to hard surfaces, such as teeth, forming a biofilm or plaque. Such colonization can eventually lead to a number of undesirable clinical side effects that include origination of caries, calcified plaque, irritation of gum tissue leading up to periodontal diseases, etc. Therefore, some of the clinical benefits of using antimicrobial agents in dental materials, such as coatings, sealants, and varnishes, are not only to kill harmful bacteria in the oral cavity but also to suppress the formation of biofilm and secondary caries on teeth and surrounding tissue.

As detailed in the Examples Section, effective amounts of certain present invention compositions (tested as coatings on polypropylene film) have provided activity against S. mutans as evaluated by the Turbidity Test Method described herein. Effective amounts of certain present invention compositions have provided Average Turbidity Ratings of less than 3.0, preferably less than 2.0, and more preferably less than 1.0, wherein a polymer coating with no antimicrobial component in the presence of bacteria had an Average Turbidity Rating of about 3.0 and a polymer coating in the absence of bacteria had an Average Turbidity Rating of 0.0.

Antimicrobial Lipid Component

The antimicrobial lipid component is that component of the composition that provides at least part of the antimicrobial activity. That is, the antimicrobial lipid component has at least some antimicrobial activity for at least one microorganism. It is generally considered the main active component of the compositions of the present invention.

In certain embodiments, the antimicrobial lipid preferably has a solubility in water of no greater than 1.0 gram per 100 grams (1.0 g/100 g) deionized water. More preferred antimicrobial lipids have a solubility in water of no greater than 0.5 g/100 g deionized water, even more preferably, no greater than 0.25 g/100 g deionized water, and even more preferably, no greater than 0.10 g/100 g deionized water. Preferred antimicrobial lipids have a solubility in deionized water of at least 100 micrograms (μg) per 100 grams deionized water, more preferably, at least 500 μg/100 g deionized water, and even more preferably, at least 1000 μg/100 g deionized water.

The antimicrobial lipids preferably have a hydrophile/lipophile balance (HLB) of at most 6.2, more preferably at most 5.8, and even more preferably at most 5.5. The antimicrobial lipids preferably have an HLB of at least 3, preferably at least 3.2, and even more preferably at least 3.4.

Preferred antimicrobial lipids are uncharged and have an alkyl or alkenyl hydrocarbon chain containing at least 7 carbon atoms.

In certain embodiments, the antimicrobial lipid component preferably includes one or more fatty acid esters of a polyhydric alcohol, fatty ethers of a polyhydric alcohol, or alkoxylated derivatives thereof (of either or both of the ester and ether), or combinations thereof. More specifically and preferably, the antimicrobial component is selected from the group consisting of a (C7-C14)saturated fatty acid ester of a polyhydric alcohol (preferably, a (C7-C12)saturated fatty acid ester of a polyhydric alcohol, and more preferably, a (C8-C12)saturated fatty acid ester of a polyhydric alcohol), a (C8-C22)unsaturated fatty acid ester of a polyhydric alcohol (preferably, a (C12-C22)unsaturated fatty acid ester of a polyhydric alcohol), a (C7-C14)saturated fatty ether of a polyhydric alcohol (preferably, a (C7-C12)saturated fatty ether of a polyhydric alcohol, and more preferably, a (C8-C12)saturated fatty ether of a polyhydric alcohol), a (C8-C22)unsaturated fatty ether of a polyhydric alcohol (preferably, a (C12-C22)unsaturated fatty ether of a polyhydric alcohol), an alkoxylated derivative thereof, and combinations thereof. Preferably, the esters and ethers are monoesters and monoethers, unless they are esters and ethers of sucrose in which case they can be monoesters, diesters, monoethers, or monoethers. Various combinations of monoesters, diesters, monoethers, and diethers can be used in a composition of the present invention.

A fatty acid ester of a polyhydric alcohol is preferably of the formula (R¹—C(O)—O)ₙ—R², wherein R¹ is the residue of a (C7-C14)saturated fatty acid (preferably, a (C7-C12)saturated fatty acid, and more preferably, a (C8-C12)saturated fatty acid), or a (C8-C22)unsaturated fatty acid (preferably, a (C12-C22)unsaturated, including polyunsaturated, fatty acid), R² is the residue of a polyhydric alcohol (typically and preferably, glycerin, propylene glycol, and sucrose, although a wide variety of others can be used including pentaerythritol, sorbitol, mannitol, xylitol, etc.), and n=1 or 2. The R² group includes at least one free hydroxyl group (preferably, residues of glycerin, propylene glycol, or sucrose). Preferred fatty acid esters of polyhydric alcohols are esters derived from C7, C8, C9, C10, C11, and C12 saturated fatty acids. For embodiments in which the polyhydric alcohol is glycerin or propylene glycol, n=1, although when it is sucrose, n=1 or 2.

Exemplary fatty acid monoesters include, but are not limited to, glycerol monoesters of lauric (monolaurin), caprylic (monocaprylin), and capric (monocaprin) acid, and propylene glycol monoesters of lauric, caprylic, and capric acid, as well as lauric, caprylic, and capric acid monoesters of sucrose. Other fatty acid monoesters include glycerin and propylene glycol monoesters of oleic (18:1), linoleic (18:2), linolenic (18:3), and arachonic (20:4) unsaturated (including polyunsaturated) fatty acids. As is generally known, 18:1, for example, means the compound has 18 carbon atoms and 1 carbon-carbon double bond. Preferred unsaturated chains have at least one unsaturated group in the cis isomer form. In certain preferred embodiments, the fatty acid monoesters that are suitable for use in the present composition include known monoesters of lauric, caprylic, and capric acid, such as that known as GML or the trade designation LAURICIDIN (the glycerol monoester of lauric acid commonly referred to as monolaurin or glycerol monolaurate), glycerol monocaprate, glycerol monocaprylate, propylene glycol monolaurate, propylene glycol monocaprate, propylene glycol monocaprylate, and combinations thereof.

Exemplary fatty acid diesters of sucrose include, but are not limited to, lauric, caprylic, and capric diesters of sucrose as well as combinations thereof.

A fatty ether of a polyhydric alcohol is preferably of the formula (R³—O)ₙ—R⁴, wherein R³ is a (C7-C14)saturated aliphatic group (preferably, a (C7-C12)saturated aliphatic group, and more preferably, a (C8-C12)saturated aliphatic group), or a (C8-C22)unsaturated aliphatic group (preferably, a (C12-C22)unsaturated, including polyunsaturated, aliphatic group), R⁴ is the residue of glycerin, sucrose, or propylene glycol, and n=1 or 2. For glycerin and propylene glycol n=1, and for sucrose n=1 or 2. Preferred fatty ethers are monoethers of (C7-C14)alkyl groups (more preferably, (C7-C12)alkyl groups, and even more preferably, (C8-C12)alkyl groups).

Exemplary fatty monoethers include, but are not limited to, laurylglyceryl ether, caprylglycerylether, caprylylglyceryl ether, laurylpropylene glycol ether, caprylpropyleneglycol ether, and caprylylpropyleneglycol ether. Other fatty monoethers include glycerin and propylene glycol monoethers of oleyl (18:1), linoleyl (18:2), linolenyl (18:3), and arachonyl (20:4) unsaturated and polyunsaturated fatty alcohols. In certain preferred embodiments, the fatty monoethers that are suitable for use in the present composition include laurylglyceryl ether, caprylglycerylether, caprylyl glyceryl ether, laurylpropylene glycol ether, caprylpropyleneglycol ether, caprylylpropyleneglycol ether, and combinations thereof. Unsaturated chains preferably have at least one unsaturated bond in the cis isomer form.

The alkoxylated derivatives of the aforementioned fatty acid esters and fatty ethers (e.g., one which is ethoxylated and/or propoxylated on the remaining alcohol group(s)) also have antimicrobial activity as long as the total alkoxylate is kept relatively low. Preferred alkoxylation levels are disclosed in U.S. Pat. No. 5,208,257 (Kabara). In the case where the esters and ethers are ethoxylated, the total moles of ethylene oxide is preferably less than 5, and more preferably less than 2.

The fatty acid esters or fatty ethers of polyhydric alcohols can be alkoxylated, preferably ethoxylated and/or propoxylated, by conventional techniques. Alkoxylating compounds are preferably selected from the group consisting of ethylene oxide, propylene oxide, and mixtures thereof, and similar oxirane compounds.

The compositions of the present invention include one or more fatty acid esters, fatty ethers, alkoxylated fatty acid esters, or alkoxylated fatty ethers at a suitable level to produce the desired result. Such compositions preferably include a total amount of such material of at least 0.01 percent by weight (wt-%), more preferably at least 0.1 wt-%, even more preferably at least 0.25 wt-%, even more preferably at least 0.5 wt-%, and even more preferably at least 1 wt-%, based on the total weight of the “ready to use” or “as used” composition. In a preferred embodiment, they are present in a total amount of no greater than 20 wt-%, more preferably no greater than 15 wt-%, even more preferably no greater than 10 wt-%, and even more preferably no greater than 5 wt-%, based on the “ready to use” or “as used” composition. Certain compositions may be higher in concentration if they are intended to be diluted prior to use.

Preferred compositions of the present invention that include one or more fatty acid monoesters, fatty monoethers, or alkoxylated derivatives thereof can also include a small amount of a di- or tri-fatty acid ester (i.e., a fatty acid di- or tri-ester), a di- or tri-fatty ether (i.e., a fatty di- or tri-ether), or alkoxylated derivative thereof. Preferably, such components are present in an amount of no more than 50 wt-%, more preferably no more than 40 wt-%, even more preferably no more than 25 wt-%, even more preferably no more than 15 wt-%, even more preferably no more than 10 wt-%, even more preferably no more than 7 wt-%, even more preferably no more than 6 wt-%, and even more preferably no more than 5 wt-%, based on the total weight of the antimicrobial lipid component. For example, for monoesters, monoethers, or alkoxylated derivatives of glycerin, preferably there is no more than 15 wt-%, more preferably no more than 10 wt-%, even more preferably no more than 7 wt-%, even more preferably no more than 6 wt-%, and even more preferably no more than 5 wt-% of a diester, diether, triester, triether, or alkoxylated derivatives thereof present, based on the total weight of the antimicrobial lipid components present in the composition. However, as will be explained in greater detail below, higher concentrations of di- and tri-esters may be tolerated in the raw material if the formulation initially includes free glycerin because of transesterification reactions.

Although in some situations it is desirable to avoid di- or tri-esters as a component of the starting materials, it is possible to use relatively pure tri-esters in the preparation of certain compositions of the present invention (for example, as a hydrophobic component) and have effective antimicrobial activity.

To achieve rapid antimicrobial activity, formulations may incorporate one or more antimicrobial lipids in the composition approaching, or preferably exceeding, the solubility limit in the hydrophobic phase. While not intended to be bound by theory, it appears that antimicrobial lipids that preferably partition into the hydrophobic component are not readily available to kill microorganisms which are in or associated with an aqueous phase in or on the tissue. In most compositions, the antimicrobial lipid is preferably incorporated in at least 60%, preferably, at least 75%, more preferably, at least 100%, and most preferably, at least 120%, of the solubility limit of the hydrophobic component at 23° C. This is conveniently determined by making the formulation without the antimicrobial lipid, separating the phases (e.g., by centrifugation or other suitable separation technique) and determining the solubility limit by addition of progressively greater levels of the antimicrobial lipid until precipitation occurs. One skilled in the art will realize that creation of supersaturated solutions must be avoided for an accurate determination.

Enhancer Component

Compositions of the present invention preferably include an enhancer (preferably a synergist) to enhance the antimicrobial activity especially against Gram negative bacteria, such as E. coli and Psuedomonas sp. The chosen enhancer preferably affects the cell envelope of the bacteria. While not bound by theory, it is presently believed that the enhancer functions by allowing the antimicrobial lipid to more easily enter the cell cytoplasm and/or by facilitating disruption of the cell envelope. The enhancer component may include an alpha-hydroxy acid, a beta-hydroxy acid, other carboxylic acids, a phenolic compound (such as certain antioxidants and parabens), a monohydroxy alcohol, a chelating agent, or a glycol ether (i.e., ether glycol). Various combinations of enhancers can be used if desired.

The alpha-hydroxy acid, beta-hydroxy acid, and other carboxylic acid enhancers are preferably present in their protonated, free acid form. It is not necessary for all of the acidic enhancers to be present in the free acid form; however, the preferred concentrations listed below refer to the amount present in the free acid form. Additional, non-alpha hydroxy acid, beta-hydroxy acid or other carboxylic acid enhancers, may be added in order to acidify the formulation or buffer it at a pH to maintain antimicrobial activity. Furthermore, the chelator enhancers that include carboxylic acid groups are preferably present with at least one, and more preferably at least two, carboxylic acid groups in their free acid form. The concentrations given below assume this to be the case.

One or more enhancers may be used in the compositions of the present invention at a suitable level to produce the desired result. In a preferred embodiment, they are present in a total amount greater than 0.01 wt-%, more preferably in an amount greater than 0.1 wt-%, even more preferably in an amount greater than 0.2 wt-%, even more preferably in an amount greater than 0.25 wt-%, and most preferably in an amount greater than 0.4 wt-% based on the total weight of the ready to use composition. In a preferred embodiment, they are present in a total amount of no greater than 20 wt-%, based on the total weight of the ready to use composition. Such concentrations typically apply to alpha-hydroxy acids, beta-hydroxy acids, other carboxylic acids, chelating agents, phenolics, ether glycols, and (C5-C10)monohydroxy alcohols. Generally, higher concentrations are needed for (C1-C4)monohydroxy alcohols, as described in greater detail below.

The alpha-hydroxy acid, beta-hydroxy acid, and other carboxylic acid enhancers, as well as chelators that include carboxylic acid groups, are preferably present in a concentration of no greater than 100 milliMoles per 100 grams of formulated composition. In most embodiments, alpha-hydroxy acid, beta-hydroxy acid, and other carboxylic acid enhancers, as well as chelators that include carboxylic acid groups, are preferably present in a concentration of no greater than 75 milliMoles per 100 grams, more preferably no greater than 50 milliMoles per 100 grams, and most preferably no greater than 25 milliMoles per 100 grams of formulated composition.

The total concentration of the enhancer component relative to the total concentration of the antimicrobial lipid component is preferably within a range of 10:1 to 1:300, and more preferably 5:1 to 1:10, on a weight basis.

An additional consideration when using an enhancer is the solubility and physical stability in the compositions. Many of the enhancers discussed herein are insoluble in hydrophobic components.

Alternatively, the enhancer may be present in excess of the solubility limit provided that the composition is physically stable. This may be achieved by utilizing a sufficiently viscous composition that stratification (e.g., settling or creaming) of the antimicrobial lipid does not appreciably occur.

Alpha-hydroxy Acids

An alpha-hydroxy acid is typically a compound represented by the formula:
R⁵(CR⁶OH)ₙCOOH
wherein: R⁵ and R⁶ are each independently H, a (C1-C8)alkyl group (straight, branched, or cyclic group), a (C6-C12)aryl group, a (C6-C12)aralkyl group, or a (C6-C12)alkaryl group (wherein the alkyl group of the aralkyl or alkaryl is straight, branched, or cyclic), wherein R⁵ and R⁶ may be optionally substituted with one or more carboxylic acid groups; and n=1-3, preferably, n=1-2.

Exemplary alpha-hydroxy acids include, but are not limited to, lactic acid, malic acid, citric acid, 2-hydroxybutanoic acid, mandelic acid, gluconic acid, glycolic acid, tartaric acid, alpha-hydroxyoctanoic acid, and alpha-hydroxycaprylic acid, as well as derivatives thereof (e.g., compounds substituted with hydroxyls, phenyl groups, hydroxyphenyl groups, alkyl groups, halogens, as well as combinations thereof). Preferred alpha-hydroxy acids include lactic acid, malic acid, and mandelic acid. These acids may be in D, L, or DL form and may be present as free acid, lactone, or partial salts thereof. All such forms are encompassed by the term “acid.” Preferably, the acids are present in the free acid form. In certain preferred embodiments, the alpha-hydroxy acids useful in the compositions of the present invention are selected from the group consisting of lactic acid, mandelic acid, and malic acid, and mixtures thereof. Other suitable alpha-hydroxy acids are described in U.S. Pat. No. 5,665,776 (Yu).

One or more alpha-hydroxy acids may be used in the compositions of the present invention at a suitable level to produce the desired result. In a preferred embodiment, they are present in a total amount of at least 0.25 wt-%, more preferably, at least 0.5 wt-%, and even more preferably, at least 1 wt-%, based on the total weight of the ready to use composition. In a preferred embodiment, they are present in a total amount of no greater than 10 wt-%, more preferably, no greater than 5 wt-%, and even more preferably, no greater than 3 wt-%, based on the total weight of the ready to use composition. Higher concentrations may become irritating.

The ratio of alpha-hydroxy acid enhancer to total antimicrobial lipid component is preferably at most 10:1, more preferably at most 5:1, and even more preferably at most 1:1. The ratio of alpha-hydroxy acid enhancer to total antimicrobial lipid component is preferably at least 1:20, more preferably at least 1:12, and even more preferably at least 1:5. Preferably the ratio of alpha-hydroxy acid enhancer to total antimicrobial lipid component is within a range of 1:12 to 1:1.

Beta-hydroxy Acids

A beta-hydroxy acid is typically a compound represented by the formula:
R⁷(CR⁸OH)ₙ(CHR⁹)ₘCOOH or 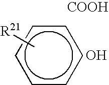
wherein: R⁷, R⁸, and R⁹ are each independently H, a (C1-C8)alkyl group (saturated straight, branched, or cyclic group), a (C6-C12)aryl group, a (C6-C12)aralkyl group, or a (C6-C12)alkaryl group (wherein the alkyl group of the aralkyl or alkaryl is straight, branched, or cyclic), wherein R⁷ and R⁸ may be optionally substituted with one or more carboxylic acid groups; m=0 or 1; n=1-3 (preferably, n=1-2); and R²¹ is H, (C1-C4)alkyl or a halogen.

Exemplary beta-hydroxy acids include, but are not limited to, salicylic acid, beta-hydroxybutanoic acid, tropic acid, 4-aminosalicylic acid, and trethocanic acid. In certain preferred embodiments, the beta-hydroxy acids useful in the compositions of the present invention are selected from the group consisting of salicylic acid, beta-hydroxybutanoic acid, and mixtures thereof. Other suitable beta-hydroxy acids are described in U.S. Pat. No. 5,665,776 (Yu).

One or more beta-hydroxy acids may be used in the compositions of the present invention at a suitable level to produce the desired result. In a preferred embodiment, they are present in a total amount of at least 0.1 wt-%, more preferably at least 0.25 wt-%, and even more preferably at least 0.5 wt-%, based on the total weight of the ready to use composition. In a preferred embodiment, they are present in a total amount of no greater than 10 wt-%, more preferably no greater than 5 wt-%, and even more preferably no greater than 3 wt-%, based on the total weight of the ready to use composition. Higher concentrations may become irritating.

The ratio of beta-hydroxy acid enhancer to total antimicrobial lipid component is preferably at most 10:1, more preferably at most 5:1, and even more preferably at most 1:1. The ratio of beta-hydroxy acid enhancer to total antimicrobial lipid component is preferably at least 1:20, more preferably at least 1:15, and even more preferably at least 1:10. Preferably the ratio of beta-hydroxy acid enhancer to total antimicrobial lipid component is within a range of 1:15 to 1:1.

In systems with low concentrations of water, or that are essentially free of water, transesterification may be the principle route of loss of the fatty acid monoester and alkoxylated derivatives of these active ingredients and loss of carboxylic acid containing enhancers may occur due to esterification. Thus, certain alpha-hydroxy acids (AHA) and beta-hydroxy acids (BHA) are particularly preferred since these are believed to be less likely to transesterify the ester antimicrobial lipid or other esters by reaction of the hydroxyl group of the AHA or BHA. For example, salicylic acid may be particularly preferred in certain formulations since the phenolic hydroxyl group is much more acidic than an aliphatic hydroxyl group and thus much less likely to react. Other particularly preferred compounds in anhydrous or low-water content formulations include lactic, mandelic, malic, citric, tartaric, and glycolic acid. Benzoic acid and substituted benzoic acids that do not include a hydroxyl group, while not hydroxy acids, are also preferred due to a reduced tendency to form ester groups.

Other Carboxylic Acids

Carboxylic acids other than alpha- and beta-carboxylic acids are suitable for use in the enhancer component. These include alkyl, aryl, aralkyl, or alkaryl carboxylic acids typically having equal to or less than 16, and often equal to or less than 12 carbon atoms. A preferred class of these can be represented by the following formula:
R¹⁰(CR¹¹₂)ₙCOOH
wherein: R¹⁰ and R¹¹ are each independently H, a (C1-C4)alkyl group (which can be a straight, branched, or cyclic group), a (C6-C12)aryl group, a (C6-C16) group containing both aryl groups and alkyl groups (which can be a straight, branched, or cyclic group), wherein R¹⁰ and R¹¹ may be optionally substituted with one or more carboxylic acid groups; and n=0-3, preferably, n=0-2. Preferably, the carboxylic acid is a (C1-C4)alkyl carboxylic acid, a (C6-C12)aralkyl carboxylic acid, or a (C6-C16)alkaryl carboxylic acid.

Exemplary acids include, but are not limited to, acetic acid, propionic acid, benzoic acid, benzylic acid, nonylbenzoic acid, p-hydroxybenzoic acid, retinoic acid, and the like. Particularly preferred is benzoic acid.

One or more carboxylic acids (other than alpha- or beta-hydroxy acids) may be used in the compositions of the present invention at a suitable level to produce the desired result. In a preferred embodiment, they are present in a total amount of at least 0.1 wt-%, more preferably at least 0.25 wt-%, even more preferably at least 0.5 wt-%, and most preferably at least 1 wt-%, based on the ready to use concentration composition. In a preferred embodiment, they are present in a total amount of no greater than 10 wt-%, more preferably no greater than 5 wt-%, and even more preferably no greater than 3 wt-%, based on the ready to use composition.

The ratio of the total concentration of carboxylic acids (other than alpha- or beta-hydroxy acids) to the total concentration of the antimicrobial lipid component is preferably within a range of 10:1 to 1:100, and more preferably 2:1 to 1:10, on a weight basis.

Chelators

A chelating agent (i.e., chelator) is typically an organic compound capable of multiple coordination sites with a metal ion in solution. Typically these chelating agents are polyanionic compounds and coordinate best with polyvalent metal ions. Exemplary chelating agents include, but are not limited to, ethylene diamine tetraacetic acid (EDTA) and salts thereof (e.g., EDTA(Na)₂, EDTA(Na)₄, EDTA(Ca), EDTA(K)₂), sodium acid pyrophosphate, acidic sodium hexametaphosphate, adipic acid, succinic acid, polyphosphoric acid, sodium acid pyrophosphate, sodium hexametaphosphate, acidified sodium hexametaphosphate, nitrilotris(methylenephosphonic acid), diethylenetriaminepentaacetic acid, ethylenebis(oxyethylenenitrilo)tetraacetic acid, glycolether diaminetetraacetic acid, ethyleneglycol-O,O′bis(2-aminoethyl)-N,N,N′,N′-tetraacetic acid (EGTA), N-(2-hydroxyethyl)ethylenediamine-N,N′,N′-triacetic acid trisodium salt (HETA), polyethylene glycol diaminetetraacetic acid, 1-hydroxyethylene, 1,1-diphosphonic acid (HEDP), and diethylenetriaminepenta-(methylenephosphonic acid). Any of these chelating agents may also be used in their partial or complete salt form. Certain carboxylic acids, particularly the alpha-hydroxy acids and beta-hydroxy acids, can also function as chelators, e.g., malic acid, ctiric, and tartaric acid.

Also included as chelators are compounds highly specific for binding ferrous and/or ferric ion such as siderophores, and iron binding proteins. Iron binding proteins include, for example, lactoferrin, and transferrin. Siderophores include, for example, enterochelin, enterobactin, vibriobactin, anguibactin, pyochelin, pyoverdin, and aerobactin.

In certain preferred embodiments, the chelating agents useful in the compositions of the present invention include those selected from the group consisting of ethylenediaminetetraacetic acid and salts thereof, succinic acid, and mixtures thereof. Preferably, either the free acid or the mono- or di-salt form of EDTA is used.

One or more chelating agents may be used in the compositions of the present invention at a suitable level to produce the desired result. In a preferred embodiment, they are present in a total amount of at least 0.01 wt-%, more preferably at least 0.05 wt-%, even more preferably at least 0.1 wt-%, and even more preferably at least 1 wt-%, based on the weight of the ready to use composition. In a preferred embodiment, they are present in a total amount of no greater than 10 wt-%, more preferably no greater than 5 wt-%, and even more preferably no greater than 1 wt-%, based on the weight of the ready to use composition.

The ratio of the total concentration of chelating agents (other than alpha- or beta-hydroxy acids) to the total concentration of the antimicrobial lipid component is preferably within a range of 10:1 to 1:100, and more preferably 1:1 to 1:10, on a weight basis.

Phenolic Compounds

A phenolic compound enhancer (i.e., a phenol or a phenol derivative) is typically a compound having the following general structure (including at least one group bonded to the ring through an oxygen): 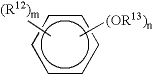
wherein: m is 0 to 3 (especially 1 to 3), n is 1 to 3 (especially 1 to 2), each R¹² independently is alkyl or alkenyl of up to 12 carbon atoms (especially up to 8 carbon atoms) optionally substituted with O in or on the chain (e.g., as a carbonyl group) or OH on the chain, and each R¹³ independently is H or alkyl or alkenyl of up to 8 carbon atoms (especially up to 6 carbon atoms) optionally substituted with O in or on the chain (e.g., as a carbonyl group) or OH on the chain, but where R¹³ is H, n preferably is 1 or 2.

Examples of phenolic enhancers include, but are not limited to, butylated hydroxy anisole, e.g., 3(2)-tert-butyl-4-methoxyphenol (BHA), 2,6-di-tert-butyl-4-methylphenol (BHT), 3,5-di-tert-butyl-4-hydroxybenzylphenol, 2,6-di-tert-4-hexylphenol, 2,6-di-tert-4-octylphenol, 2,6-di-tert-4-decylphenol, 2,6-di-tert-butyl-4-ethylphenol, 2,6-di-tert-4-butylphenol, 2,5-di-tert-butylphenol, 3,5-di-tert-butylphenol, 4,6-di-tert-butyl-resorcinol, methyl paraben (4-hydroxybenzoic acid methyl ester), ethyl paraben, propyl paraben, butyl paraben, as well as combinations thereof. A preferred group of the phenolic compounds is the phenol species having the general structure shown above where R¹³═H and where R¹² is alkyl or alkenyl of up to 8 carbon atoms, and n is 1, 2, or 3, especially where at least one R¹² is butyl and particularly tert-butyl, and especially the non-toxic members thereof. Some of the preferred phenolic synergists are BHA, BHT, methyl paraben, ethyl paraben, propyl paraben, and butyl paraben as well as combinations of these.

One or more phenolic compounds may be used in the compositions of the present invention at a suitable level to produce the desired result. The concentrations of the phenolic compounds in medical-grade compositions may vary widely, but as little as 0.001 wt-%, based on the total weight of the composition, can be effective when the above-described esters are present within the above-noted ranges. In a preferred embodiment, they are present in a total amount of at least 0.01 wt-%, more preferably at least 0.10 wt-%, and even more preferably at least 0.25 wt-%, based on the ready to use composition. In a preferred embodiment, they are present in a total amount of no greater than 8 wt-%, more preferably no greater than 4 wt-%, and even more preferably no greater than 2 wt-%, based on the ready to use composition.

It is preferred that the ratio of the total phenolic concentration to the total concentration of the antimicrobial lipid component be within a range of 10:1 to 1:300, and more preferably within a range of 1:1 to 1:10, on a weight basis.

The above-noted concentrations of the phenolics are normally observed unless concentrated formulations for subsequent dilution are intended. On the other hand, the minimum concentration of the phenolics and the antimicrobial lipid components to provide an antimicrobial effect will vary with the particular application.

Monohydroxy Alcohols

An additional enhancer class includes monohydroxy alcohols having 1-10 carbon atoms. This includes the lower (i.e., C1-C4) monohydroxy alcohols (e.g., methanol, ethanol, isopropanol, and butanol) as well as longer chain (i.e., C5-C10) monohydroxy alcohols (e.g., isobutanol, t-butanol, octanol, and decanol). Other useful alcohols include phenoxyethanol, benzyl alcohol, and menthol. In certain preferred embodiments, the alcohols useful in the compositions of the present invention are selected from the group consisting of methanol, ethanol, isopropyl alcohol, and mixtures thereof.

One or more alcohols may be used in the compositions of the present invention at a suitable level to produce the desired result. In a preferred embodiment, the short chain (i.e., C1-C4) alcohols are present in a total amount of at least 10 wt-%, even more preferably at least 15 wt-%, even more preferably at least 20 wt-%, and even more preferably at least 25 wt-%, based on the total weight of the ready to use composition.

In a preferred embodiment, the (C1-C4)alcohols are present in a total amount of no greater than 90 wt-%, more preferably no greater than 70 wt-%, even more preferably no greater than 60 wt-%, and even more preferably no greater than 50 wt-%, based on the total weight of the ready to use composition.

For certain applications, lower alcohols may not be preferred due to the strong odor and potential for stinging and irritation. This can occur especially at higher levels. In applications where stinging or burning is a concern, the concentration of (C1-C4)alcohols is preferably less than 20 wt-%, more preferably less than 15 wt-%.

In another preferred embodiment longer chain (i.e., C5-C10)alcohols are present in a total amount of at least 0.1 wt-%, more preferably at least 0.25 wt-%, and even more preferably at least 0.5 wt-%, and most preferably at least 1.0%, based on the ready to use composition. In a preferred embodiment, the (C5-C10)alcohols are present in a total amount of no greater than 10 wt-%, more preferably no greater than 5 wt-%, and even more preferably no greater than 2 wt-%, based on the total weight of the ready to use composition.

Ether Glycols

An additional enhancer class includes ether glycols. Exemplary ether glycols include those of the formula:
R′—O—(CH₂CHR″O)ₙ(CH₂CHR″O)H
wherein R′═H, a (C1-C8)alkyl, a (C6-C12)aryl group, a (C6-C12)aralkyl group, or a (C6-C12)alkaryl group; and each R″ is independently ═H, methyl, or ethyl; and n=0-5, preferably 1-3. Examples include 2-phenoxyethanol, dipropylene glycol, triethylene glycol, the line of products available under the trade designation DOWANOL DB (di(ethylene glycol) butyl ether), DOWANOL DPM (di(propylene glycol)monomethyl ether), and DOWANOL TPnB (tri(propylene glycol) monobutyl ether), as well as many others available from Dow Chemical, Midland, Mich.

One or more ether glycols may be used in the compositions of the present invention at a suitable level to produce the desired result. In a preferred embodiment, they are present in a total amount of at least 0.01 wt-%, based on the total weight of the ready to use composition. In a preferred embodiment, they are present in a total amount of no greater than 20 wt-%, based on the total weight of the ready to use composition.

Surfactants

Compositions of the present invention can optionally include one or more surfactants. In some embodiments, the presence of a surfactant may be used to emulsify the composition and to help wet the surface and/or to aid in contacting the microorganisms. As used herein the term “surfactant” means an amphiphile (a molecule possessing both polar and nonpolar regions which are covalently bound) capable of reducing the surface tension of water and/or the interfacial tension between water and an immiscible liquid. The term is meant to include soaps, detergents, emulsifiers, surface active agents, and the like. The surfactant can be cationic, anionic, nonionic, or amphoteric. This includes a wide variety of conventional surfactants. Combinations of various surfactants can be used if desired.

Certain ethoxylated surfactants can reduce or eliminate the antimicrobial efficacy of the antimicrobial lipid component. The exact mechanism of this is not known and not all ethoxylated surfactants display this negative effect. For example, poloxamer (polyethylene oxide/polypropylene oxide) surfactants have been shown to be compatible with the antimicrobial lipid component, but ethoxylated sorbitan fatty acid esters such as those sold under the trade name TWEEN by ICI have not been compatible. It should be noted that these are broad generalizations and the activity could be formulation dependent.

It should be noted that certain antimicrobial lipds are amphiphiles and may be surface active. For example, certain antimicrobial alkyl monoglycerides described herein are surface active. For certain embodiments of the invention, the antimicrobial lipid component is considered distinct from a “surfactant” component.

Preferred surfactants are those that have an HLB (i.e., hydrophile to lipophile balance) of at least 4 and more preferably at least 8. Even more preferred surfactants have an HLB of at least 12. Most preferred surfactants have an HLB of at least 15; however, lower HLB surfactants are still useful in compositions described herein.

Examples of the various classes of surfactants are described below. In certain preferred embodiments, the surfactants useful in the compositions of the present invention are selected from the group consisting of sulfonates, sulfates, phosphonates, phosphates, poloxamer (polyethylene oxide/polypropylene oxide block copolymers), cationic surfactants, and mixtures thereof. In certain more preferred embodiments, the surfactants useful in the compositions of the present invention are selected from the group consisting of sulfonates, sulfates, phosphates, and mixtures thereof.

One or more surfactants may be used in the compositions of the present invention at a suitable level to produce the desired result. In a preferred embodiment, they are present in a total amount of at least 0.1 wt-%, more preferably at least 0.5 wt-%, and even more preferably at least 1.0 wt-%, based on the total weight of the ready to use composition.

Surfactants may be present in a total amount of no greater than 10 wt-%, more preferably no greater than 5 wt-%, even more preferably no greater than 3 wt-%, and even more preferably no greater than 2 wt-%, based on the total weight of the ready to use composition. The ratio of the total concentration of surfactant to the total concentration of the antimicrobial lipid component is preferably within a range of 5:1 to 1:100, more preferably 3:1 to 1:10, and most preferably 2:1 to 1:3, on a weight basis.

Cationic Surfactants

Exemplary cationic surfactants include, but are not limited to, salts of optionally polyoxyalkylenated primary, secondary, or tertiary fatty amines; quaternary ammonium salts such as tetraalkylammonium, alkylamidoalkyltrialkylammonium, trialkylbenzylammonium, trialkylhydroxyalkylammonium, or alkylpyridinium halides (preferably chlorides or bromides) as well as other anionic counterions, such as but not limited to, alkyl sulfates, such as but not limited to, methosulfate and ethosulfate; imidazoline derivatives; amine oxides of a cationic nature (e.g., at an acidic pH); and mixtures thereof.

In certain embodiments, the cationic surfactants useful in the compositions of the present invention are selected from the group consisting of tetralkyl ammonium, trialkylbenzylammonium, and alkylpyridinium halides as well as other anionic counterions, such as but not limited to, C1-C4 alkyl sulfates, such as but not limited to, methosulfate and ethosulfate, and mixtures thereof.

Amine Oxide Surfactants

Amine oxide surfactants, which can be cationic or nonionic depending on the pH (e.g., cationic at lower pH and nonionic at higher pH). Amine oxide surfactants including alkyl and alkylamidoalkyldialkylamine oxides of the following formula:
(R¹⁴)₃—N→O
wherein R¹⁴ is a (C1-C30)alkyl group (preferably a (C1-C14)alkyl group) or a (C6-C18)aralklyl or alkaryl group, wherein any of these groups can be optionally substituted in or on the chain by N—, O—, or S-containing groups such as amide, ester, hydroxyl, and the like. Each R¹⁴ may be the same or different provided at least one R¹⁴ group includes at least eight carbons. Optionally, the R¹⁴ groups can be joined to form a heterocyclic ring with the nitrogen to form surfactants such as amine oxides of alkyl morpholine, alkyl piperazine, and the like. Preferably two R¹⁴ groups are methyl and one R¹⁴ group is a (C12-C16)alkyl or alkylamidopropyl group. Examples of amine oxide surfactants include those commercially available under the trade designations AMMONYX LO, LMDO, and CO, which are lauryldimethylamine oxide, laurylamidopropyldimethylamine oxide, and cetyl amine oxide, all from Stepan Company of Northfield, Ill.
Anionic Surfactants

Exemplary anionic surfactants include, but are not limited to, sarcosinates, glutamates, alkyl sulfates, sodium or potassium alkyleth sulfates, ammonium alkyleth sulfates, ammonium laureth-n-sulfates, laureth-n-sulfates, isethionates, glycerylether sulfonates, sulfosuccinates, alkylglyceryl ether sulfonates, alkyl phosphates, aralkyl phosphates, alkylphosphonates, and aralkylphosphonates. These anionic surfactants may have a metal or organic ammonium counterion. In certain preferred embodiments, the anionic surfactants useful in the compositions of the present invention are selected from the group consisting of:

1. Sulfonates and Sulfates. Suitable anionic surfactants include sulfonates and sulfates such as alkyl sulfates, alkylether sulfates, alkyl sulfonates, alkylether sulfonates, alkylbenzene sufonates, alkylbenzene ether sulfates, alkylsulfoacetates, secondary alkane sulfonates, secondary alkylsulfates, and the like. Many of these can be represented by the formulas:
R¹⁴—(OCH₂CH₂)ₙ(OCH(CH₃)CH₂)ₚ-(Ph)ₐ-(OCH₂CH₂)ₘ—(O)_{b}—SO₃⁻M⁺
and
R¹⁴—CH[SO₃-M⁺]-R¹⁵
wherein: a and b=0 or 1; n, p, and m=0-100 (preferably 0-20, and more preferably 0-10); R¹⁴ is defined as above provided at least one R¹⁴ or R¹⁵ is at least C8; R¹⁵ is a (C1-C12)alkyl group (saturated straight, branched, or cyclic group) that may be optionally substituted by N, O, or S atoms or hydroxyl, carboxyl, amide, or amine groups; Ph=phenyl; and M is a cationic counterion such as H, Na, K, Li, ammonium, or a protonated tertiary amine such as triethanolamine or a quaternary ammonium group.

In the formula above, the ethylene oxide groups (i.e., the “n” and “m” groups) and propylene oxide groups (i.e., the “p” groups) can occur in reverse order as well as in a random, sequential, or block arrangement. Preferably for this class, R¹⁴ includes an alkylamide group such as R¹⁶—C(O)N(CH₃)CH₂CH₂— as well as ester groups such as —OC(O)—CH₂— wherein R¹⁶ is a (C8-C22)alkyl group (branched, straight, or cyclic group). Examples include, but are not limited to: alkyl ether sulfonates such as lauryl ether sulfates such as POLYSTEP B12 (n=3-4, M=sodium) and B22 (n=12, M=ammonium) available from Stepan Company, Northfield, Ill. and sodium methyl taurate (available under the trade designation NIKKOL CMT30 from Nikko Chemicals Co., Tokyo, Japan); secondary alkane sulfonates such as Hostapur SAS which is a Sodium (C14-C17)secondary alkane sulfonates (alpha-olefin sulfonates) available from Clariant Corp., Charlotte, N.C.; methyl-2-sulfoalkyl esters such as sodium methyl-2-sulfo(C12-16)ester and disodium 2-sulfo(C12-C16)fatty acid available from Stepan Company under the trade designation ALPHASTEP PC-48; alkylsulfoacetates and alkylsulfosuccinates available as sodium laurylsulfoacetate (under the trade designation LANTHANOL LAL) and disodiumlaurethsulfosuccinate (STEPANMILD SL3), both from Stepan Company; alkylsulfates such as ammoniumlauryl sulfate commercially available under the trade designation STEPANOL AM from Stepan Company; dialkylsulfosuccinates such as dioctylsodiumsulfosuccinate available as Aerosol OT from Cytec Industries. Hydrotropes such as DOWFAX hydrotrope from Dow chemical or other diphenyl oxide surfactants may also be used.

2. Phosphates and Phosphonates. Suitable anionic surfactants also include phosphates such as alkyl phosphates, alkylether phosphates, aralkylphosphates, and aralkylether phosphates. Many may be represented by the formula:
[R¹⁴-(Ph)ₐ-O(CH₂CH₂O)ₙ(CH₂CH(CH₃)O)ₚ]_{q}—P(O)[O⁻M⁺]ᵣ
wherein: Ph, R¹⁴, a, n, p, and M are defined above; r is 0-2; and q=1-3; with the proviso that when q=1, r=2, and when q=2, r=1, and when q=3, r=0. As above, the ethylene oxide groups (i.e., the “n” groups) and propylene oxide groups (i.e., the “p” groups) can occur in reverse order as well as in a random, sequential, or block arrangement. Examples include a mixture of mono-, di- and tri-(alkyltetraglycolether)-o-phosphoric acid esters generally referred to as trilaureth-4-phosphate commercially available under the trade designation HOSTAPHAT 340KL from Clariant Corp., Charlotte, N.C., as well as PPG-5 ceteth 10 phosphate available under the trade designation CRODAPHOS SG from Croda Inc., Parsipanny, N.J., and mixtures thereof.
Amphoteric Surfactants

Surfactants of the amphoteric type include surfactants having tertiary amine groups, which may be protonated, as well as quaternary amine containing zwitterionic surfactants. Such surfactants include:

1. Ammonium Carboxylate Amphoterics. This class of surfactants can be represented by the following formula:
R¹⁷—(C(O)—NH)ₐ—R¹⁸—N⁺(R¹⁹)₂—R²⁰—COO⁻
wherein: a=0 or 1; R¹⁷ is a (C7-C21)alkyl group (saturated straight, branched, or cyclic group), a (C6-C22)aryl group, or a (C6-C22)aralkyl or alkaryl group (saturated straight, branched, or cyclic alkyl group), wherein R¹⁷ may be optionally substituted with one or more N, O, or S atoms, or one or more hydroxyl, carboxyl, amide, or amine groups; R¹⁹ is H or a (C1-C8)alkyl group (saturated straight, branched, or cyclic group), wherein R¹⁹ may be optionally substituted with one or more N, O, or S atoms, or one or more hydroxyl, carboxyl, amine groups, a (C6-C9)aryl group, or a (C6-C9)aralkyl or alkaryl group; and R¹⁸ and R²⁰ are each independently a (C1-C10)alkylene group that may be the same or different and may be optionally substituted with one or more N, O, or S atoms, or one or more hydroxyl or amine groups.

In other embodiments, in the formula above, R¹⁷ is a (C1-C18)alkyl group, R¹⁹ is a (C1-C2)alkyl group preferably substituted with a methyl or benzyl group and most preferably with a methyl group. When R¹⁹ is H it is understood that the surfactant at higher pH values could exist as a tertiary amine with a cationic counterion such as Na, K, Li, or a quaternary amine group.

Examples of such amphoteric surfactants include, but are not limited to: certain betaines such as cocobetaine and cocamidopropyl betaine (commercially available under the trade designations MACKAM CB-35 and MACKAM L from McIntyre Group Ltd., University Park, Ill.); monoacetates such as sodium lauroamphoacetate; diacetates such as disodium lauroamphoacetate; and amino- and alkylamino-propionates such as lauraminopropionic acid (commercially available under the trade designations MACKAM 1L, MACKAM 2L, and MACKAM 151L, respectively, from McIntyre Group Ltd.).

2. Ammonium Sulfonate Amphoterics. This class of amphoteric surfactants are often referred to as “sultaines” or “sulfobetaines” and can be represented by the following formula
R¹⁷—(C(O)—NH)ₐ—R¹⁸—N⁺(R¹⁹)₂—R²⁰—SO₃⁻
wherein R¹⁷—R²⁰ and “a” are defined above. Examples include cocamidopropylhydroxysultaine (commercially available as MACKAM 50-SB from McIntyre Group Ltd.). The sulfoamphoterics may be preferred over the carboxylate amphoterics since the sulfonate group will remain ionized at much lower pH values.
Nonionic Surfactants

Exemplary nonionic surfactants include, but are not limited to, alkyl glucosides, alkyl polyglucosides, polyhydroxy fatty acid amides, sucrose esters, esters of fatty acids and polyhydric alcohols, fatty acid alkanolamides, ethoxylated fatty acids, ethoxylated aliphatic acids, ethoxylated fatty alcohols (e.g., octyl phenoxy polyethoxyethanol available under the trade name TRITON X-100 and nonyl phenoxy poly(ethyleneoxy) ethanol available under the trade name NONIDET P-40, both from Sigma, St. Louis, Mo.), ethoxylated and/or propoxylated aliphatic alcohols (e.g., that available under the trade name BRIJ from ICI, Wilmington, Del.), ethoxylated glycerides, ethoxylated/propoxylated block copolymers such as PLURONIC and TETRONIC surfactants available from BASF, ethoxylated cyclic ether adducts, ethoxylated amide and imidazoline adducts, ethoxylated amine adducts, ethoxylated mercaptan adducts, ethoxylated condensates with alkyl phenols, ethoxylated nitrogen-based hydrophobes, ethoxylated polyoxypropylenes, polymeric silicones, fluorinated surfactants (e.g., those available under the trade names FLUORAD-FS 300 from 3M Company, St. Paul, Minn., and ZONYL from DuPont de Nemours Co., Wilmington, Del.), and polymerizable (reactive) surfactants (e.g., SAM 211 (alkylene polyalkoxy sulfate) surfactant available under the trade name MAZON from PPG Industries, Inc., Pittsburgh, Pa.). In certain preferred embodiments, the nonionic surfactants useful in the compositions of the present invention are selected from the group consisting of Poloxamers such as PLURONIC from BASF, sorbitan fatty acid esters, and mixtures thereof. A particularly preferred nonionic surfactant is P65 poloxamer (polyethylene oxide capped polypropylene oxide having a EO/PO mole ratio of 1 and a molecular weight of approximately 3400) available from BASF Wyandotte Corp., Parsippany, N.J.

Water-Dispersible Polymeric Film-Former

In some embodiments, water-dispersible polymeric film-formers as disclosed herein include a repeating unit that includes a polar or polarizable group as described herein below. In certain embodiments, the water-dispersible polymeric film-formers also include a repeating unit that includes a fluoride releasing group (preferably containing a tetrafluoroborate anion), a repeating unit that includes a hydrophobic hydrocarbon group, a repeating unit that includes a graft polysiloxane chain, a repeating unit that includes a hydrophobic fluorine-containing group, a repeating unit that includes a modulating group, or combinations thereof, as described herein below. In some embodiments, the polymer optionally includes a reactive group (e.g., ethylenically unsaturated groups, epoxy groups, or silane moieties capable of undergoing a condensation reaction). In some embodiments, the water-dispersible polymeric film-formers include two or more different types of repeating units, such as those described above. Exemplary water-dispersible polymeric film-formers are disclosed, for example, in U.S. Pat. No. 5,468,477 (Kumar et al.), U.S. Pat. No. 5,525,648 (Aasen et al.), U.S. Pat. No. 5,607,663 (Rozzi et al.), U.S. Pat. No. 5,662,887 (Rozzi et al.), U.S. Pat. No. 5,725,882 (Kumar et al.), U.S. Pat. No. 5,866,630 (Mitra et al.), U.S. Pat. No. 5,876,208 (Mitra et al.), U.S. Pat. No. 5,888,491 (Mitra et al.), U.S. Pat. No. 6,312,668 (Mitra et al.) and U.S. Pat. Publication No. 2004/0185013 (Mitra et al.).

Polar or Polarizable Groups

Repeating units including a polar or polarizable group are derived from vinylic monomers such as acrylates, methacrylates, crotonates, itaconates, and the like. The polar groups can be acidic, basic or salt. These groups can also be ionic or neutral.

Examples of polar or polarizable groups include neutral groups such as hydroxy, thio, substituted and unsubstituted amido, cyclic ethers (such as oxanes, oxetanes, furans and pyrans), basic groups (such as phosphines and amines, including primary, secondary, tertiary amines), acidic groups (such as oxy acids, and thiooxyacids of C, S, P, B), ionic groups (such as quarternary ammonium, carboxylate salt, sulfonic acid salt and the like), and the precursors and protected forms of these groups. Additionally, a polar or polarizable group could be a macromonomer. More specific examples of such groups follow.

Polar or polarizable groups may be derived from mono- or multifunctional carboxyl group containing molecules represented by the general formula:
CH₂═CR²²G-(COOH)_{d}
where R²²═H, methyl, ethyl, cyano, carboxy or carboxymethyl, d=1-5, and G is a bond or a hydrocarbyl radical linking group containing from 1-12 carbon atoms of valence d+1 and optionally substituted with and/or interrupted with a substituted or unsubstituted heteroatom (such as O, S, N and P). Optionally, this unit may be provided in its salt form. The preferred monomers in this class are acrylic acid, methacrylic acid, itaconic acid, and N-acryloyl glycine.

Polar or polarizable groups may, for example, be derived from mono- or multifunctional hydroxy group containing molecules represented by the general formula:
CH₂═CR²²—CO-L-R²³—(OH)_{d}
where R²²═H, methyl, ethyl, cyano, carboxy or carboxyalkyl, L=O or NH, d=1-5, and R²³ is a hydrocarbyl radical of valence d+1 containing from 1-12 carbon atoms. The preferred monomers in this class are hydroxyethyl(meth)acrylate, hydroxypropyl(meth)acrylate, hydroxybutyl(meth)acrylate, glycerol mono(meth)acrylate, tris(hydroxymethyl)ethane monoacrylate, pentaerythritol mono(meth)acrylate, N-hydroxymethyl(meth)acrylamide, hydroxyethyl(meth)acrylamide, and hydroxypropyl(meth)acrylamide.

Polar or polarizable groups may alternatively be derived from mono- or multifunctional amino group containing molecules of the general formula:
CH₂═CR²²—CO-L-R²³—(NR²⁴R²⁵)_{d}
where R²², L, R²³, and d are as defined above and R²⁴ and R²⁵ are H or alkyl groups of 1-12 carbon atoms or together they constitute a carbocyclic or heterocyclic group. Preferred monomers of this class are aminoethyl(meth)acrylate, aminopropyl(meth)acrylate, N,N-dimethylaminoethyl(meth)acrylate, N,N-diethylaminoethyl(meth)acrylate, N,N-dimethylaminopropyl(meth)acrylamide, N-isopropylaminopropyl(meth)acrylamide, and 4-methyl-1-acryloyl-piperazine.

Polar or polarizable groups may also be derived from alkoxy substituted (meth)acrylates or (meth)acrylamides such as methoxyethyl(meth)acrylate, 2-(2-ethoxyethoxy)ethyl(meth)acrylate, polyethylene glycol mono(meth)acrylate or polypropylene glycol mono(meth)acrylate.

Polar or polarizable groups may be derived from substituted or unsubstituted ammonium monomers of the general formula:
CH₂═CR²²—CO-L-R²³—(NR²⁴R²⁵R²⁶)_{d}Q⁻
where R²², R²³, R²⁴, R²⁵, L and d are as defined above, and where R²⁶ is H or alkyl of 1-12 carbon atoms and Q⁻ is an organic or inorganic anion. Preferred examples of such monomers include 2-N,N,N-trimethylammonium ethyl(meth)acrylate, 2-N,N,N-triethylammonium ethyl(meth)acrylate, 3-N,N,N-trimethylammonium propyl(meth)acrylate, N(2-N′,N′,N′-trimethylammonium)ethyl(meth)acrylamide, N-(dimethyl hydroxyethyl ammonium) propyl(meth)acrylamide, or combinations thereof, where the counterion may include fluoride, chloride, bromide, acetate, propionate, laurate, palmitate, stearate, or combinations thereof. The monomer can also be N,N-dimethyl diallyl ammonium salt of an organic or inorganic counterion.

Ammonium group containing polymers can also be prepared by using as the polar or polarizable group any of the amino group containing monomer described above, and acidifying the resultant polymers with organic or inorganic acid to a pH where the pendant amino groups are substantially protonated. Totally substituted ammonium group containing polymers may be prepared by alkylating the above described amino polymers with alkylating groups, the method being commonly known in the art as the Menschutkin reaction.

Polar or polarizable groups can also be derived from sulfonic acid group containing monomers, such as vinyl sulfonic acid, styrene sulfonic acid, 2-acrylamido-2-methyl propane sulfonic acid, allyloxybenzene sulfonic acid, and the like. Alternatively, polar or polarizable groups may be derived from phosphorous acid or boron acid group-containing monomers. These monomers may be used in the protonated acid form as monomers and the corresponding polymers obtained may be neutralized with an organic or inorganic base to give the salt form of the polymers.

Preferred repeating units of a polar or polarizable group include acrylic acid, itaconic acid, N-isopropylacrylamide, or combinations thereof.

In certain embodiments, the water-dispersible polymeric film-formers disclosed herein also include a repeating unit that includes a fluoride releasing group. A preferred fluoride releasing group includes tetrafluoroborate anions as disclosed, for example, in U.S. Pat. No. 4,871,786 (Aasen et al.). A preferred repeating unit of a fluoride releasing group includes trimethylammoniumethyl methacrylate.

Hydrophobic Hydrocarbon Groups

In certain embodiments, the water-dispersible polymeric film-formers disclosed herein also include a repeating unit that includes a hydrophobic hydrocarbon group. An exemplary hydrophobic hydrocarbon group is derived from an ethylenically unsaturated preformed hydrocarbon moiety having a weight average molecular weight greater than 160. Preferably the hydrocarbon moiety has a molecular weight of at least 160. Preferably the hydrocarbon moiety has a molecular weight of at most 100,000, and more preferably at most 20,000. The hydrocarbon moiety may be aromatic or non-aromatic in nature, and optionally may contain partially or fully saturated rings. Preferred hydrophobic hydrocarbon moieties are dodecyl and octadecyl acrylates and methacrylates. Other preferred hydrophobic hydrocarbon moieties include macromonomers of the desired molecular weights prepared from polymerizable hydrocarbons, such as ethylene, styrene, alpha-methyl styrene, vinyltoluene, and methyl methacrylate.

Hydrophobic Fluorine Containing Groups

In certain embodiments, the water-dispersible polymeric film-formers disclosed herein also include a repeating unit that includes a hydrophobic fluorine containing group. Exemplary repeating units of hydrophobic fluorine-containing groups include acrylic or methacrylic acid esters of 1,1-dihydroperfluoroalkanols and homologs: CF₃(CF₂)ₓCH₂OH and CF₃(CF₂)ₓ(CH₂)_{y}OH, where x is zero to 20 and y is at least 1 up to 10; ω-hydrofluoroalkanols (HCF₂(CF₂)ₓ(CH₂)_{y}OH), where x is 0 to 20 and y is at least 1 up to 10; fluoroalkylsulfonamido alcohols; cyclic fluoroalkyl alcohols; and CF₃(CF₂CF₂O)_{q}(CF₂O)ₓ(CH₂)_{y}OH, where q is 2 to 20 and greater than x, x is 0 to 20, and y is at least 1 up to 10.

Preferred repeating units of a hydrophobic fluorine-containing group include 2-(methyl(nonafluorobutyl)sulfonyl)amino)ethyl acrylate, 2-(methyl(nonafluorobutyl)sulfonyl)amino)ethyl methacrylate, or combinations thereof.

Graft Polysiloxane Chains

In certain embodiments, the water-dispersible polymeric film-formers disclosed herein also include a repeating unit that includes a graft polysiloxane chain. The graft polysiloxane chain is derived from an ethylenically unsaturated preformed organosiloxane chain. The molecular weight of this unit is generally above 500. Preferred repeating units of a graft polysiloxane chain include a silicone macromer.

Monomers used to provide the graft polysiloxane chain of this invention are terminally functional polymers having a single functional group (vinyl, ethylenically unsaturated, acryloyl, or methacryloyl group) and are sometimes termed macromonomers or “macromers.” Such monomers are known and may be prepared by methods as disclosed, for example, in U.S. Pat. No. 3,786,116 (Milkovich et al.) and U.S. Pat. No. 3,842,059 (Milkovich et al.). The preparation of polydimethylsiloxane macromonomer and subsequent copolymerization with vinyl monomer have been described in several papers by Y. Yamashita et al., Polymer J., 14, 913 (1982); ACS Polymer Preprints, 25 (1), 245 (1984); Makromol. Chem., 185, 9 (1984).

Modulating Groups

In certain embodiments, the water-dispersible polymeric film-formers disclosed herein also include a repeating unit that includes a modulating group. Exemplary modulating groups are derived from acrylate or methacrylate or other vinyl polymerizable starting monomers and optionally contain functionalities that modulate properties such as glass transition temperature, solubility in the carrier medium, hydrophilic-hydrophobic balance and the like.

Examples of modulating groups include the lower to intermediate methacrylic acid esters of 1-12 carbon straight, branched or cyclic alcohols. Other examples of modulating groups include styrene, vinyl esters, vinyl chloride, vinylidene chloride, acryloyl monomers and the like.

In some embodiments, water-dispersible polymeric film-formers as disclosed herein include amide-functional polymers, such as polymers that include monomeric units derived from N-isopropylacrylamide (e.g., polymerized or copolymerized N-isopropylacrylamide) and reactive polymers that include a polymeric backbone having one or more unsaturated pendant groups and a plurality of pendant groups of the formula —C(O)NHCH(CH₃)₂ attached to the backbone. Preferably the pendant ethylenically unsaturated group includes a (meth)acrylate group. Such polymers are described in U.S. patent application Ser. No. 10/626,341 (Ali et al.; filed Jul. 24, 2003) and U.S. Pat. Publication No. 2004/0185013 (Mitra et al.).

In other embodiments, water-dispersible polymeric film-formers as disclosed herein include siloxane polymers functionalized with pendant moieties that include anionic groups, for example, carboxylic acid groups, including dicarboxy acid groups. Such polymers (especially dicarboxy-functionalized siloxane plymers) are described in U.S. Pat. Publication No. 2003/0211051 (Majeti et al.).

Preferred film-formers are acrylate-based copolymers and urethane polymers such as the AVALURE series of compounds (e.g., AC-315 and UR-450), and carbomer-based polymers such as the CARBOPOL series of polymers (e.g., 940NF), all available from Noveon, Inc., Cleveland, Ohio.

In some embodiments of the present invention, a dental composition can include a water-dispersible polymeric film-former component and a hardenable component separate from (i.e., different than) the water-dispersible polymeric film-former component.

Hardenable Component

Dental compositions of the present invention may also include a hardenable (e.g., polymerizable) component, thereby forming hardenable (e.g., polymerizable) compositions. The hardenable component can include a wide variety of chemistries, such as ethylenically unsaturated compounds (with or without acid functionality), epoxy (oxirane) resins, vinyl ethers, photopolymerization systems, redox cure systems, glass ionomer cements, polyethers, polysiloxanes, and the like. In some embodiments, the compositions can be hardened (e.g., polymerized by conventional photopolymerization and/or chemical polymerization techniques) prior to applying the dental material. In other embodiments, the compositions can be hardened (e.g., polymerized by conventional photopolymerization and/or chemical polymerization techniques) after applying the dental material.

In certain embodiments, the compositions are photopolymerizable, i.e., the compositions contain a photoinitiator (i.e., a photoinitiator system) that upon irradiation with actinic radiation initiates the polymerization (or hardening) of the composition. Such photopolymerizable compositions can be free radically polymerizable or cationically polymerizable. In other embodiments, the compositions are chemically hardenable, i.e., the compositions contain a chemical initiator (i.e., initiator system) that can polymerize, cure, or otherwise harden the composition without dependence on irradiation with actinic radiation. Such chemically hardenable compositions are sometimes referred to as “self-cure” compositions and may include glass ionomer cements (e.g., conventional and resin-modified glass ionomer cements), redox cure systems, and combinations thereof.

Suitable photopolymerizable components that can be used in the dental compositions of the present invention include, for example, epoxy resins (which contain cationically active epoxy groups), vinyl ether resins (which contain cationically active vinyl ether groups), ethylenically unsaturated compounds (which contain free radically active unsaturated groups, e.g., acrylates and methacrylates), and combinations thereof. Also suitable are polymerizable materials that contain both a cationically active functional group and a free radically active functional group in a single compound. Examples include epoxy-functional acrylates, epoxy-functional methacrylates, and combinations thereof.

Ethylenically Unsaturated Compounds

Compositions of the present invention may include one or more hardenable components in the form of ethylenically unsaturated compounds with or without acid functionality, thereby forming hardenable compositions.

Suitable hardenable compositions may include hardenable components (e.g., photopolymerizable compounds) that include ethylenically unsaturated compounds (which contain free radically active unsaturated groups). Examples of useful ethylenically unsaturated compounds include acrylic acid esters, methacrylic acid esters, hydroxy-functional acrylic acid esters, hydroxy-functional methacrylic acid esters, and combinations thereof.

The compositions (e.g., photopolymerizable compositions) may include compounds having free radically active functional groups that may include monomers, oligomers, and polymers having one or more ethylenically unsaturated group. Suitable compounds contain at least one ethylenically unsaturated bond and are capable of undergoing addition polymerization. Such free radically polymerizable compounds include mono-, di- or poly-(meth)acrylates (i.e., acrylates and methacrylates) such as, methyl (meth)acrylate, ethyl acrylate, isopropyl methacrylate, n-hexyl acrylate, stearyl acrylate, allyl acrylate, glycerol triacrylate, ethyleneglycol diacrylate, diethyleneglycol diacrylate, triethyleneglycol dimethacrylate, 1,3-propanediol di(meth)acrylate, trimethylolpropane triacrylate, 1,2,4-butanetriol trimethacrylate, 1,4-cyclohexanediol diacrylate, pentaerythritol tetra(meth)acrylate, sorbitol hexacrylate, tetrahydrofurfuryl (meth)acrylate, bis[1-(2-acryloxy)]-p-ethoxyphenyldimethylmethane, bis[1-(3-acryloxy-2-hydroxy)]-p-propoxyphenyldimethylmethane, ethoxylated bisphenolA di(meth)acrylate, and trishydroxyethyl-isocyanurate trimethacrylate; (meth)acrylamides (i.e., acrylamides and methacrylamides) such as (meth)acrylamide, methylene bis-(meth)acrylamide, and diacetone (meth)acrylamide; urethane (meth)acrylates; the bis-(meth)acrylates of polyethylene glycols (preferably of molecular weight 200-500), copolymerizable mixtures of acrylated monomers such as those in U.S. Pat. No. 4,652,274 (Boettcher et al.), acrylated oligomers such as those of U.S. Pat. No. 4,642,126 (Zador et al.), and poly(ethylenically unsaturated) carbamoyl isocyanurates such as those disclosed in U.S. Pat. No. 4,648,843 (Mitra); and vinyl compounds such as styrene, diallyl phthalate, divinyl succinate, divinyl adipate and divinyl phthalate. Other suitable free radically polymerizable compounds include siloxane-functional (meth)acrylates as disclosed, for example, in WO-00/38619 (Guggenberger et al.), WO-01/92271 (Weinmann et al.), WO-01/07444 (Guggenberger et al.), WO-00/42092 (Guggenberger et al.) and fluoropolymer-functional (meth)acrylates as disclosed, for example, in U.S. Pat. No. 5,076,844 (Fock et al.), U.S. Pat. No. 4,356,296 (Griffith et al.), EP-0373 384 (Wagenknecht et al.), EP-0201 031 (Reiners et al.), and EP-0201 778 (Reiners et al.). Mixtures of two or more free radically polymerizable compounds can be used if desired.

The hardenable component may also contain hydroxyl groups and ethylenically unsaturated groups in a single molecule. Examples of such materials include hydroxyalkyl (meth)acrylates, such as 2-hydroxyethyl (meth)acrylate and 2-hydroxypropyl (meth)acrylate; glycerol mono- or di-(meth)acrylate; trimethylolpropane mono- or di-(meth)acrylate; pentaerythritol mono-, di-, and tri-(meth)acrylate; sorbitol mono-, di-, tri-, tetra-, or penta-(meth)acrylate; and 2,2-bis[4-(2-hydroxy-3-methacryloxypropoxy)phenyl]propane (bisGMA). Suitable ethylenically unsaturated compounds are also available from a wide variety of commercial sources, such as Sigma-Aldrich, St. Louis, Mo. Mixtures of ethylenically unsaturated compounds can be used if desired.

In certain embodiments hardenable components include PEGDMA (polyethyleneglycol dimethacrylate having a molecular weight of approximately 400), bisGMA, UDMA (urethane dimethacrylate), GDMA (glycerol dimethacrylate), TEGDMA (triethyleneglycol dimethacrylate), bisEMA6 as described in U.S. Pat. No. 6,030,606 (Holmes), and NPGDMA (neopentylglycol dimethacrylate). Various combinations of the hardenable components can be used if desired.

Preferably, compositions of the present invention include at least 5% by weight, more preferably at least 10% by weight, and most preferably at least 15% by weight ethylenically unsaturated compounds, based on the total weight of the unfilled composition. Preferably, compositions of the present invention include at most 95% by weight, more preferably at most 90% by weight, and most preferably at most 80% by weight ethylenically unsaturated compounds, based on the total weight of the unfilled composition.

Preferably, compositions of the present invention include ethylenically unsaturated compounds without acid functionality. Preferably, compositions of the present invention include at least 5% by weight (wt-%), more preferably at least 10% by weight, and most preferably at least 15% by weight ethylenically unsaturated compounds without acid functionality, based on the total weight of the unfilled composition. Preferably, compositions of the present invention include at most 95% by weight, more preferably at most 90% by weight, and most preferably at most 80% by weight ethylenically unsaturated compounds without acid functionality, based on the total weight of the unfilled composition.

Ethylenically Unsaturated Compounds with Acid Functionality

Compositions of the present invention may include one or more hardenable components in the form of ethylenically unsaturated compounds with acid functionality, thereby forming hardenable compositions.

As used herein, ethylenically unsaturated compounds with acid functionality is meant to include monomers, oligomers, and polymers having ethylenic unsaturation and acid and/or acid-precursor functionality. Acid-precursor functionalities include, for example, anhydrides, acid halides, and pyrophosphates. The acid functionality can include carboxylic acid functionality, phosphoric acid functionality, phosphonic acid functionality, sulfonic acid functionality, or combinations thereof.

Ethylenically unsaturated compounds with acid functionality include, for example, α,β-unsaturated acidic compounds such as glycerol phosphate mono(meth)acrylates, glycerol phosphate di(meth)acrylates, hydroxyethyl (meth)acrylate (e.g., HEMA) phosphates, bis((meth)acryloxyethyl) phosphate, ((meth)acryloxypropyl) phosphate, bis((meth)acryloxypropyl) phosphate, bis((meth)acryloxy)propyloxy phosphate, (meth)acryloxyhexyl phosphate, bis((meth)acryloxyhexyl) phosphate, (meth)acryloxyoctyl phosphate, bis((meth)acryloxyoctyl) phosphate, (meth)acryloxydecyl phosphate, bis((meth)acryloxydecyl) phosphate, caprolactone methacrylate phosphate, citric acid di- or tri-methacrylates, poly(meth)acrylated oligomaleic acid, poly(meth)acrylated polymaleic acid, poly(meth)acrylated poly(meth)acrylic acid, poly(meth)acrylated polycarboxyl-polyphosphonic acid, poly(meth)acrylated polychlorophosphoric acid, poly(meth)acrylated polysulfonate, poly(meth)acrylated polyboric acid, and the like, may be used as components in the hardenable component system. Also monomers, oligomers, and polymers of unsaturated carbonic acids such as (meth)acrylic acids, aromatic (meth)acrylated acids (e.g., methacrylated trimellitic acids), and anhydrides thereof can be used. Certain preferred compositions of the present invention include an ethylenically unsaturated compound with acid functionality having at least one P—OH moiety.

Certain of these compounds are obtained, for example, as reaction products between isocyanatoalkyl (meth)acrylates and carboxylic acids. Additional compounds of this type having both acid-functional and ethylenically unsaturated components are described in U.S. Pat. No. 4,872,936 (Engelbrecht) and U.S. Pat. No. 5,130,347 (Mitra). A wide variety of such compounds containing both the ethylenically unsaturated and acid moieties can be used. Mixtures of such compounds can be used if desired.

Additional ethylenically unsaturated compounds with acid functionality include, for example, polymerizable bisphosphonic acids as disclosed for example, in U.S. Pat. Publication No. 2004/0206932 (Abuelyaman et al.); AA:ITA:IEM (copolymer of acrylic acid:itaconic acid with pendent methacrylate made by reacting AA:ITA copolymer with sufficient 2-isocyanatoethyl methacrylate to convert a portion of the acid groups of the copolymer to pendent methacrylate groups as described, for example, in Example 11 of U.S. Pat. No. 5,130,347 (Mitra)); and those recited in U.S. Pat. No. 4,259,075 (Yamauchi et al.), U.S. Pat. No. 4,499,251 (Omura et al.), U.S. Pat. No. 4,537,940 (Omura et al.), U.S. Pat. No. 4,539,382 (Omura et al.), U.S. Pat. No. 5,530,038 (Yamamoto et al.), U.S. Pat. No. 6,458,868 (Okada et al.), and European Pat. Application Publication Nos. EP 712,622 (Tokuyama Corp.) and EP 1,051,961 (Kuraray Co., Ltd.).

Preferably, the compositions of the present invention include at least 1% by weight, more preferably at least 3% by weight, and most preferably at least 5% by weight ethylenically unsaturated compounds with acid functionality, based on the total weight of the unfilled composition. Preferably, compositions of the present invention include at most 80% by weight, more preferably at most 70% by weight, and most preferably at most 60% by weight ethylenically unsaturated compounds with acid functionality, based on the total weight of the unfilled composition.

Epoxy (Oxirane) or Vinyl Ether Compounds

The hardenable compositions of the present invention may include one or more hardenable components in the form of epoxy (oxirane) compounds (which contain cationically active epoxy groups) or vinyl ether compounds (which contain cationically active vinyl ether groups), thereby forming hardenable compositions.

The epoxy or vinyl ether monomers can be used alone as the hardenable component in a dental composition or in combination with other monomer classes, e.g., ethylenically unsaturated compounds as described herein, and can include as part of their chemical structures aromatic groups, aliphatic groups, cycloaliphatic groups, and combinations thereof.

Examples of epoxy (oxirane) compounds include organic compounds having an oxirane ring that is polymerizable by ring opening. These materials include monomeric epoxy compounds and epoxides of the polymeric type and can be aliphatic, cycloaliphatic, aromatic or heterocyclic. These compounds generally have, on the average, at least 1 polymerizable epoxy group per molecule, in some embodiments at least 1.5, and in other embodiments at least 2 polymerizable epoxy groups per molecule. The polymeric epoxides include linear polymers having terminal epoxy groups (e.g., a diglycidyl ether of a polyoxyalkylene glycol), polymers having skeletal oxirane units (e.g., polybutadiene polyepoxide), and polymers having pendent epoxy groups (e.g., a glycidyl methacrylate polymer or copolymer). The epoxides may be pure compounds or may be mixtures of compounds containing one, two, or more epoxy groups per molecule. The “average” number of epoxy groups per molecule is determined by dividing the total number of epoxy groups in the epoxy-containing material by the total number of epoxy-containing molecules present.

These epoxy-containing materials may vary from low molecular weight monomeric materials to high molecular weight polymers and may vary greatly in the nature of their backbone and substituent groups. Illustrative of permissible substituent groups include halogens, ester groups, ethers, sulfonate groups, siloxane groups, carbosilane groups, nitro groups, phosphate groups, and the like. The molecular weight of the epoxy-containing materials may vary from 58 to 100,000 or more.

Suitable epoxy-containing materials useful as the resin system reactive components in the present invention are listed in U.S. Pat. No. 6,187,836 (Oxman et al.) and U.S. Pat. No. 6,084,004 (Weinmann et al.).

Other suitable epoxy resins useful as the resin system reactive components include those which contain cyclohexene oxide groups such as epoxycyclohexanecarboxylates, typified by 3,4-epoxycyclohexylmethyl-3,4-epoxycyclohexanecarboxylate, 3,4-epoxy-2-methylcyclohexylmethyl-3,4-epoxy-2-methylcyclohexane carboxylate, and bis(3,4-epoxy-6-methylcyclohexyl-methyl) adipate. For a more detailed list of useful epoxides of this nature, reference is made to U.S. Pat. No. 6,245,828 (Weinmann et al.) and U.S. Pat. No. 5,037,861 (Crivello et al.); and U.S. Pat. Publication No. 2003/035899 (Klettke et al.).

Other epoxy resins that may be useful in the compositions of this invention include glycidyl ether monomers. Examples are glycidyl ethers of polyhydric phenols obtained by reacting a polyhydric phenol with an excess of chlorohydrin such as epichlorohydrin (e.g., the diglycidyl ether of 2,2-bis-(2,3-epoxypropoxyphenol)propane). Further examples of epoxides of this type are described in U.S. Pat. No. 3,018,262 (Schroeder), and in “Handbook of Epoxy Resins” by Lee and Neville, McGraw-Hill Book Co., New York (1967).

Other suitable epoxides useful as the resin system reactive components are those that contain silicon, useful examples of which are described in International Pat. Publication No. WO 01/51540 (Klettke et al.).

Additional suitable epoxides useful as the resin system reactive components include octadecylene oxide, epichlorohydrin, styrene oxide, vinyl cyclohexene oxide, glycidol, glycidylmethacrylate, diglycidyl ether of Bisphenol A and other commercially available epoxides, as provided in U.S. Ser. No. 10/719,598 (Oxman et al.; filed Nov. 21, 2003).

Blends of various epoxy-containing materials are also contemplated. Examples of such blends include two or more weight average molecular weight distributions of epoxy-containing compounds, such as low molecular weight (below 200), intermediate molecular weight (200 to 10,000) and higher molecular weight (above 10,000). Alternatively or additionally, the epoxy resin may contain a blend of epoxy-containing materials having different chemical natures, such as aliphatic and aromatic, or functionalities, such as polar and non-polar.

Other types of useful hardenable components having cationically active functional groups include vinyl ethers, oxetanes, spiro-orthocarbonates, spiro-orthoesters, and the like.

If desired, both cationically active and free radically active functional groups may be contained in a single molecule. Such molecules may be obtained, for example, by reacting a di- or poly-epoxide with one or more equivalents of an ethylenically unsaturated carboxylic acid. An example of such a material is the reaction product of UVR-6105 (available from Union Carbide) with one equivalent of methacrylic acid. Commercially available materials having epoxy and free-radically active functionalities include the CYCLOMER series, such as CYCLOMER M-100, M-101, or A-200 available from Daicel Chemical, Japan, and EBECRYL-3605 available from Radcure Specialties, UCB Chemicals, Atlanta, Ga.

The cationically curable components may further include a hydroxyl-containing organic material. Suitable hydroxyl-containing materials may be any organic material having hydroxyl functionality of at least 1, and preferably at least 2. Preferably, the hydroxyl-containing material contains two or more primary or secondary aliphatic hydroxyl groups (i.e., the hydroxyl group is bonded directly to a non-aromatic carbon atom). The hydroxyl groups can be terminally situated, or they can be pendent from a polymer or copolymer. The molecular weight of the hydroxyl-containing organic material can vary from very low (e.g., 32) to very high (e.g., one million or more). Suitable hydroxyl-containing materials can have low molecular weights (i.e., from 32 to 200), intermediate molecular weights (i.e., from 200 to 10,000, or high molecular weights (i.e., above 10,000). As used herein, all molecular weights are weight average molecular weights.

The hydroxyl-containing materials may be non-aromatic in nature or may contain aromatic functionality. The hydroxyl-containing material may optionally contain heteroatoms in the backbone of the molecule, such as nitrogen, oxygen, sulfur, and the like. The hydroxyl-containing material may, for example, be selected from naturally occurring or synthetically prepared cellulosic materials. The hydroxyl-containing material should be substantially free of groups which may be thermally or photolytically unstable; that is, the material should not decompose or liberate volatile components at temperatures below 100° C. or in the presence of actinic light which may be encountered during the desired photopolymerization conditions for the polymerizable compositions.

Suitable hydroxyl-containing materials useful in the present invention are listed in U.S. Pat. No. 6,187,836 (Oxman et al.).

The hardenable component(s) may also contain hydroxyl groups and cationically active functional groups in a single molecule. An example is a single molecule that includes both hydroxyl groups and epoxy groups.

Glass Ionomers

The hardenable compositions of the present invention may include glass ionomer cements such as conventional glass ionomer cements that typically employ as their main ingredients a homopolymer or copolymer of an ethylenically unsaturated carboxylic acid (e.g., poly acrylic acid, copoly (acrylic, itaconic acid), and the like), a fluoroaluminosilicate (“FAS”) glass, water, and a chelating agent such as tartaric acid. Conventional glass ionomers (i.e., glass ionomer cements) typically are supplied in powder/liquid formulations that are mixed just before use. The mixture will undergo self-hardening in the dark due to an ionic reaction between the acidic repeating units of the polycarboxylic acid and cations leached from the glass.

The glass ionomer cements may also include resin-modified glass ionomer (“RMGI”) cements. Like a conventional glass ionomer, an RMGI cement employs an FAS glass. However, the organic portion of an RMGI is different. In one type of RMGI, the polycarboxylic acid is modified to replace or end-cap some of the acidic repeating units with pendent curable groups and a photoinitiator is added to provide a second cure mechanism, e.g., as described in U.S. Pat. No. 5,130,347 (Mitra). Acrylate or methacrylate groups are usually employed as the pendant curable group. In another type of RMGI, the cement includes a polycarboxylic acid, an acrylate or methacrylate-functional monomer and a photoinitiator, e.g., as in Mathis et al., “Properties of a New Glass Ionomer/Composite Resin Hybrid Restorative”, Abstract No. 51, J. Dent Res., 66:113 (1987) and as in U.S. Pat. No. 5,063,257 (Akahane et al.), U.S. Pat. No. 5,520,725 (Kato et al.), U.S. Pat. No. 5,859,089 (Qian), U.S. Pat. No. 5,925,715 (Mitra) and U.S. Pat. No. 5,962,550 (Akahane et al.). In another type of RMGI, the cement may include a polycarboxylic acid, an acrylate or methacrylate-functional monomer, and a redox or other chemical cure system, e.g., as described in U.S. Pat. No. 5,154,762 (Mitra et al.), U.S. Pat. No. 5,520,725 (Kato et al.), and U.S. Pat. No. 5,871,360 (Kato). In another type of RMGI, the cement may include various monomer-containing or resin-containing components as described in U.S. Pat. No. 4,872,936 (Engelbrecht), U.S. Pat. No. 5,227,413 (Mitra), U.S. Pat. No. 5,367,002 (Huang et al.), and U.S. Pat. No. 5,965,632 (Orlowski). RMGI cements are preferably formulated as powder/liquid or paste/paste systems, and contain water as mixed and applied. The compositions are able to harden in the dark due to the ionic reaction between the acidic repeating units of the polycarboxylic acid and cations leached from the glass, and commercial RMGI products typically also cure on exposure of the cement to light from a dental curing lamp. RMGI cements that contain a redox cure system and that can be cured in the dark without the use of actinic radiation are described in U.S. Pat. No. 6,765,038 (Mitra).

Polyethers or Polysiloxanes (i.e., Silicones)

Dental impression materials are typically based on polyether or polysiloxane (i.e. silicone) chemistry. Polyether materials typically consist of a two-part system that includes a base component (e.g., a polyether with ethylene imine rings as terminal groups) and a catalyst (or accelerator) component (e.g., an aryl sulfonate as a cross-linking agent). Polysiloxane materials also typically consist of a two-part system that includes a base component (e.g., a polysiloxane, such as a dimethylpolysiloxane, of low to moderately low molecular weight) and a catalyst (or accelerator) component (e.g., a low to moderately low molecular weight polymer with vinyl terminal groups and chloroplatinic acid catalyst in the case of addition silicones; or a liquid that consists of stannous octanoate suspension and an alkyl silicate in the case of condensation silicones). Both systems also typically contain a filler, a plasticizer, a thickening agent, a coloring agent, or mixtures thereof. Exemplary polyether impression materials include those described in, for example, U.S. Pat. No. 6,127,449 (Bissinger et al.); U.S. Pat. No. 6,395,801 (Bissinger et al.); and U.S. Pat. No. 5,569,691 (Guggenberger et al.). Exemplary polysiloxane impression materials and related polysiloxane chemistry are described in, for example, U.S. Pat. No. 6,121,362 (Wanek et al.) and U.S. Pat. No. 6,566,413 Weinmann et al.), and EP Pat. Publication No. 1 475 069 A (Bissinger et al.).

Examples of commercial polyether and polysiloxane impression materials include, but are not limited to, IMPREGUM Polyether Materials, PERMADYNE Polyether Materials, EXPRESS Vinyl Polysiloxane Materials, DIMENSION Vinyl Polysiloxane Materials, and IMPRINT Vinyl Polysiloxane Materials; all available from 3M ESPE (St. Paul, Minn.). Other exemplary polyether, polysiloxane (silicones), and polysulfide impression materials are discussed in the following reference: Restorative Dental Materials, Tenth Edition, edited by Robert G. Craig and Marcus L. Ward, Mosby-Year Book, Inc., St. Louis, Mo., Chapter 11 (Impression Materials).

Photoinitiator Systems

In certain embodiments, the compositions of the present invention are photopolymerizable, i.e., the compositions contain a photopolymerizable component and a photoinitiator (i.e., a photoinitiator system) that upon irradiation with actinic radiation initiates the polymerization (or hardening) of the composition. Such photopolymerizable compositions can be free radically polymerizable or cationically polymerizable.

Suitable photoinitiators (i.e., photoinitiator systems that include one or more compounds) for polymerizing free radically photopolymerizable compositions include binary and tertiary systems. Typical tertiary photoinitiators include an iodonium salt, a photosensitizer, and an electron donor compound as described in U.S. Pat. No. 5,545,676 (Palazzotto et al.). Preferred iodonium salts are the diaryl iodonium salts, e.g., diphenyliodonium chloride, diphenyliodonium hexafluorophosphate, diphenyliodonium tetrafluoroborate, and tolylcumyliodonium tetrakis(pentafluorophenyl)borate. Preferred photosensitizers are monoketones and diketones that absorb some light within a range of 400 nm to 520 nm (preferably, 450 nm to 500 nm). More preferred compounds are alpha diketones that have some light absorption within a range of 400 nm to 520 nm (even more preferably, 450 to 500 nm). Preferred compounds are camphorquinone, benzil, furil, 3,3,6,6-tetramethylcyclohexanedione, phenanthraquinone, 1-phenyl-1,2-propanedione and other 1-aryl-2-alkyl-1,2-ethanediones, and cyclic alpha diketones. Most preferred is camphorquinone. Preferred electron donor compounds include substituted amines, e.g., ethyl dimethylaminobenzoate. Other suitable tertiary photoinitiator systems useful for photopolymerizing cationically polymerizable resins are described, for example, in U.S. Pat. No. 6,765,036 (Dede et al.).

Other suitable photoinitiators for polymerizing free radically photopolymerizable compositions include the class of phosphine oxides that typically have a functional wavelength range of 380 nm to 1200 nm. Preferred phosphine oxide free radical initiators with a functional wavelength range of 380 nm to 450 nm are acyl and bisacyl phosphine oxides such as those described in U.S. Pat. No. 4,298,738 (Lechtken et al.), U.S. Pat. No. 4,324,744 (Lechtken et al.), U.S. Pat. No. 4,385,109 (Lechtken et al.), U.S. Pat. No. 4,710,523 (Lechtken et al.), and U.S. Pat. No. 4,737,593 (Ellrich et al.), U.S. Pat. No. 6,251,963 (Kohler et al.); and EP Application No. 0 173 567 A2 (Ying).

Commercially available phosphine oxide photoinitiators capable of free-radical initiation when irradiated at wavelength ranges of greater than 380 nm to 450 nm include bis(2,4,6-trimethylbenzoyl)phenyl phosphine oxide (IRGACURE 819, Ciba Specialty Chemicals, Tarrytown, N.Y.), bis(2,6-dimethoxybenzoyl)-(2,4,4-trimethylpentyl) phosphine oxide (CGI 403, Ciba Specialty Chemicals), a 25:75 mixture, by weight, of bis(2,6-dimethoxybenzoyl)-2,4,4-trimethylpentyl phosphine oxide and 2-hydroxy-2-methyl-1-phenylpropan-1-one (IRGACURE 1700, Ciba Specialty Chemicals), a 1:1 mixture, by weight, of bis(2,4,6-trimethylbenzoyl)phenyl phosphine oxide and 2-hydroxy-2-methyl-1-phenylpropane-1-one (DAROCUR 4265, Ciba Specialty Chemicals), and ethyl 2,4,6-trimethylbenzylphenyl phosphinate (LUCIRIN LR8893X, BASF Corp., Charlotte, N.C.).

Typically, the phosphine oxide initiator is present in the photopolymerizable composition in catalytically effective amounts, such as from 0.1 weight percent to 5.0 weight percent, based on the total weight of the composition.

Tertiary amine reducing agents may be used in combination with an acylphosphine oxide. Illustrative tertiary amines useful in the invention include ethyl 4-(N,N-dimethylamino)benzoate and N,N-dimethylaminoethyl methacrylate. When present, the amine reducing agent is present in the photopolymerizable composition in an amount from 0.1 weight percent to 5.0 weight percent, based on the total weight of the composition. Useful amounts of other initiators are well known to those of skill in the art.

Suitable photoinitiators for polymerizing cationically photopolymerizable compositions include binary and tertiary systems. Typical tertiary photoinitiators include an iodonium salt, a photosensitizer, and an electron donor compound as described in EP 0 897 710 (Weinmann et al.); in U.S. Pat. No. 5,856,373 (Kaisaki et al.), U.S. Pat. No. 6,084,004 (Weinmann et al.), U.S. Pat. No. 6,187,833 (Oxman et al.), and U.S. Pat. No. 6,187,836 (Oxman et al.); and in U.S. Pat. No. 6,765,036 (Dede et al.).

Suitable iodonium salts include tolylcumyliodonium tetrakis(pentafluorophenyl)borate, tolylcumyliodonium tetrakis(3,5-bis(trifluoromethyl)-phenyl)borate, and the diaryl iodonium salts, e.g., diphenyliodonium chloride, diphenyliodonium hexafluorophosphate, diphenyliodonium hexafluoroantimonate, and diphenyliodonium tetrafluoroboarate. Suitable photosensitizers are monoketones and diketones that absorb some light within a range of 450 nm to 520 nm (preferably, 450 nm to 500 nm). More suitable compounds are alpha diketones that have some light absorption within a range of 450 nm to 520 nm (even more preferably, 450 nm to 500 nm). Preferred compounds are camphorquinone, benzil, furil, 3,3,6,6-tetramethylcyclohexanedione, phenanthraquinone and other cyclic alpha diketones. Most preferred is camphorquinone. Suitable electron donor compounds include substituted amines, e.g., ethyl 4-(dimethylamino)benzoate and 2-butoxyethyl 4-(dimethylamino)benzoate; and polycondensed aromatic compounds (e.g. anthracene).

The initiator system is present in an amount sufficient to provide the desired rate of hardening (e.g., polymerizing and/or crosslinking). For a photoinitiator, this amount will be dependent in part on the light source, the thickness of the layer to be exposed to radiant energy, and the extinction coefficient of the photoinitiator. Preferably, the initiator system is present in a total amount of at least 0.01 wt-%, more preferably, at least 0.03 wt-%, and most preferably, at least 0.05 wt-%, based on the weight of the composition. Preferably, the initiator system is present in a total amount of no more than 10 wt-%, more preferably, no more than 5 wt-%, and most preferably, no more than 2.5 wt-%, based on the weight of the composition.

Redox Initiator Systems

In certain embodiments, the compositions of the present invention are chemically hardenable, i.e., the compositions contain a chemically hardenable component and a chemical initiator (i.e., initiator system) that can polymerize, cure, or otherwise harden the composition without dependence on irradiation with actinic radiation. Such chemically hardenable compositions are sometimes referred to as “self-cure” compositions and may include glass ionomer cements, resin-modified glass ionomer cements, redox cure systems, and combinations thereof.

The chemically hardenable compositions may include redox cure systems that include a hardenable component (e.g., an ethylenically unsaturated polymerizable component) and redox agents that include an oxidizing agent and a reducing agent. Suitable hardenable components, redox agents, optional acid-functional components, and optional fillers that are useful in the present invention are described in U.S. Pat. Publication Nos. 2003/0166740 (Mitra et al.) and 2003/0195273 (Mitra et al.).

The reducing and oxidizing agents should react with or otherwise cooperate with one another to produce free-radicals capable of initiating polymerization of the resin system (e.g., the ethylenically unsaturated component). This type of cure is a dark reaction, that is, it is not dependent on the presence of light and can proceed in the absence of light. The reducing and oxidizing agents are preferably sufficiently shelf-stable and free of undesirable colorization to permit their storage and use under typical dental conditions. They should be sufficiently miscible with the resin system (and preferably water-soluble) to permit ready dissolution in (and discourage separation from) the other components of the hardenable composition.

Useful reducing agents include ascorbic acid, ascorbic acid derivatives, and metal complexed ascorbic acid compounds as described in U.S. Pat. No. 5,501,727 (Wang et al.); amines, especially tertiary amines, such as 4-tert-butyl dimethylaniline; aromatic sulfinic salts, such as p-toluenesulfinic salts and benzenesulfinic salts; thioureas, such as 1-ethyl-2-thiourea, tetraethyl thiourea, tetramethyl thiourea, 1,1-dibutyl thiourea, and 1,3-dibutyl thiourea; and mixtures thereof. Other secondary reducing agents may include cobalt (II) chloride, ferrous chloride, ferrous sulfate, hydrazine, hydroxylamine (depending on the choice of oxidizing agent), salts of a dithionite or sulfite anion, and mixtures thereof. Preferably, the reducing agent is an amine.

Suitable oxidizing agents will also be familiar to those skilled in the art, and include but are not limited to persulfuric acid and salts thereof, such as sodium, potassium, ammonium, cesium, and alkyl ammonium salts. Additional oxidizing agents include peroxides such as benzoyl peroxides, hydroperoxides such as cumyl hydroperoxide, t-butyl hydroperoxide, and amyl hydroperoxide, as well as salts of transition metals such as cobalt (III) chloride and ferric chloride, cerium (IV) sulfate, perboric acid and salts thereof, permanganic acid and salts thereof, perphosphoric acid and salts thereof, and mixtures thereof.

It may be desirable to use more than one oxidizing agent or more than one reducing agent. Small quantities of transition metal compounds may also be added to accelerate the rate of redox cure. In some embodiments it may be preferred to include a secondary ionic salt to enhance the stability of the polymerizable composition as described in U.S. Pat. Publication No. 2003/0195273 (Mitra et al.).

The reducing and oxidizing agents are present in amounts sufficient to permit an adequate free-radical reaction rate. This can be evaluated by combining all of the ingredients of the hardenable composition except for the optional filler, and observing whether or not a hardened mass is obtained.

Preferably, the reducing agent is present in an amount of at least 0.01% by weight, and more preferably at least 0.1% by weight, based on the total weight (including water) of the components of the hardenable composition. Preferably, the reducing agent is present in an amount of no greater than 10% by weight, and more preferably no greater than 5% by weight, based on the total weight (including water) of the components of the hardenable composition.

Preferably, the oxidizing agent is present in an amount of at least 0.01% by weight, and more preferably at least 0.10% by weight, based on the total weight (including water) of the components of the hardenable composition. Preferably, the oxidizing agent is present in an amount of no greater than 10% by weight, and more preferably no greater than 5% by weight, based on the total weight (including water) of the components of the hardenable composition.

The reducing or oxidizing agents can be microencapsulated as described in U.S. Pat. No. 5,154,762 (Mitra et al.). This will generally enhance shelf stability of the hardenable composition, and if necessary permit packaging the reducing and oxidizing agents together. For example, through appropriate selection of an encapsulant, the oxidizing and reducing agents can be combined with an acid-functional component and optional filler and kept in a storage-stable state. Likewise, through appropriate selection of a water-insoluble encapsulant, the reducing and oxidizing agents can be combined with an FAS glass and water and maintained in a storage-stable state.

A redox cure system can be combined with other cure systems, e.g., with a hardenable composition such as described U.S. Pat. No. 5,154,762 (Mitra et al.).

Fillers

The compositions of the present invention can also contain fillers. Fillers may be selected from one or more of a wide variety of materials suitable for incorporation in compositions used for dental applications, such as fillers currently used in dental restorative compositions, and the like.

The filler is preferably finely divided. The filler can have a unimodial or polymodial (e.g., bimodal) particle size distribution. Preferably, the maximum particle size (the largest dimension of a particle, typically, the diameter) of the filler is less than 20 micrometers, more preferably less than 10 micrometers, and most preferably less than 5 micrometers. Preferably, the average particle size of the filler is less than 0.1 micrometer, and more preferably less than 0.075 micrometer.

The filler can be an inorganic material. It can also be a crosslinked organic material that is insoluble in the resin system (i.e., the hardenable components), and is optionally filled with inorganic filler. The filler should in any event be nontoxic and suitable for use in the mouth. The filler can be radiopaque or radiolucent. The filler typically is substantially insoluble in water.

Examples of suitable inorganic fillers are naturally occurring or synthetic materials including, but not limited to: quartz (i.e., silica, SiO₂); nitrides (e.g., silicon nitride); glasses and fillers derived from, for example, Zr, Sr, Ce, Sb, Sn, Ba, Zn, and Al; feldspar; borosilicate glass; kaolin; talc; zirconia; titania; low Mohs hardness fillers such as those described in U.S. Pat. No. 4,695,251 (Randklev); and submicron silica particles (e.g., pyrogenic silicas such as those available under the trade designations AEROSIL, including “OX 50,” “130,” “150” and “200” silicas from Degussa Corp., Akron, Ohio and CAB-O-SIL M5 silica from Cabot Corp., Tuscola, Ill.). Examples of suitable organic filler particles include filled or unfilled pulverized polycarbonates, polyepoxides, and the like.

Preferred non-acid-reactive filler particles are quartz (i.e., silica), submicron silica, zirconia, submicron zirconia, and non-vitreous microparticles of the type described in U.S. Pat. No. 4,503,169 (Randklev). Mixtures of these non-acid-reactive fillers are also contemplated, as well as combination fillers made from organic and inorganic materials.

The filler can also be an acid-reactive filler. Suitable acid-reactive fillers include metal oxides, glasses, and metal salts. Typical metal oxides include barium oxide, calcium oxide, magnesium oxide, and zinc oxide. Typical glasses include borate glasses, phosphate glasses, and fluoroaluminosilicate (“FAS”) glasses. FAS glasses are particularly preferred. The FAS glass typically contains sufficient elutable cations so that a hardened dental composition will form when the glass is mixed with the components of the hardenable composition. The glass also typically contains sufficient elutable fluoride ions so that the hardened composition will have cariostatic properties. The glass can be made from a melt containing fluoride, alumina, and other glass-forming ingredients using techniques familiar to those skilled in the FAS glassmaking art. The FAS glass typically is in the form of particles that are sufficiently finely divided so that they can conveniently be mixed with the other cement components and will perform well when the resulting mixture is used in the mouth.

Generally, the average particle size (typically, diameter) for the FAS glass is no greater than 12 micrometers, typically no greater than 10 micrometers, and more typically no greater than 5 micrometers as measured using, for example, a sedimentation analyzer. Suitable FAS glasses will be familiar to those skilled in the art, and are available from a wide variety of commercial sources, and many are found in currently available glass ionomer cements such as those commercially available under the trade designations VITREMER, VITREBOND, RELY X LUTING CEMENT, RELY X LUTING PLUS CEMENT, PHOTAC-FIL QUICK, KETAC-MOLAR, and KETAC-FIL PLUS (3M ESPE Dental Products, St. Paul, Minn.), FUJI II LC and FUJI IX (G-C Dental Industrial Corp., Tokyo, Japan) and CHEMFIL Superior (Dentsply International, York, Pa.). Mixtures of fillers can be used if desired.

The surface of the filler particles can also be treated with a coupling agent in order to enhance the bond between the filler and the resin. The use of suitable coupling agents include gamma-methacryloxypropyltrimethoxysilane, gamma-mercaptopropyltriethoxysilane, gamma-aminopropyltrimethoxysilane, and the like. Silane-treated zirconia-silica (ZrO₂—SiO₂) filler, silane-treated silica filler, silane-treated zirconia filler, and combinations thereof are especially preferred in certain embodiments.

Other suitable fillers are disclosed in U.S. Pat. No. 6,387,981 (Zhang et al.) and U.S. Pat. No. 6,572,693 (Wu et al.) as well as International Publication Nos. WO 01/30305 (Zhang et al.), WO 01/30306 (Windisch et al.), WO 01/30307 (Zhang et al.), and WO 03/063804 (Wu et al.). Filler components described in these references include nanosized silica particles, nanosized metal oxide particles, and combinations thereof. Nanofillers are also described in U.S. patent application Ser. No. 10/847,781 (Kangas et al.); Ser. No. 10/847,782 (Kolb et al.); Ser. No. 10/847,803 (Craig et al.); and Ser. No. 10/847,805 (Budd et al.) all four of which were filed on May 17, 2004. These applications, in summary, describe the following nanofiller containing compositions:

U.S. patent application Ser. No. 10/847,781 (Kangas et al.) describes stable ionomer compositions (e.g., glass ionomer) containing nanofillers that provide the compositions with improved properties over previous ionomer compositions. In one embodiment, the composition is a hardenable dental composition comprising a polyacid (e.g., a polymer having a plurality of acidic repeating groups); an acid-reactive filler; at least 10 percent by weight nanofiller or a combination of nanofillers each having an average particle size no more than 200 nanometers; water; and optionally a polymerizable component (e.g., an ethylenically unsaturated compound, optionally with acid functionality).

U.S. patent application Ser. No. 10/847,782 (Kolb et al.) describes stable ionomer (e.g., glass ionomer) compositions containing nanozirconia fillers that provide the compositions with improved properties, such as ionomer systems that are optically translucent and radiopaque. The nanozirconia is surface modified with silanes to aid in the incorporation of the nanozirconia into ionomer compositions, which generally contain a polyacid that might otherwise interact with the nanozirconia causing coagulation or aggregation resulting in undesired visual opacity. In one aspect, the composition can be a hardenable dental composition including a polyacid; an acid-reactive filler; a nanozirconia filler having a plurality of silane-containing molecules attached onto the outer surface of the zirconia particles; water; and optionally a polymerizable component (e.g., an ethylenically unsaturated compound, optionally with acid functionality).

U.S. patent application Ser. No. 10/847,803 (Craig et al.) describes stable ionomer compositions (e.g., glass ionomers) containing nanofillers that provide the compositions with enhanced optical translucency. In one embodiment, the composition is a hardenable dental composition including a polyacid (e.g., a polymer having a plurality of acidic repeating groups); an acid-reactive filler; a nanofiller; an optional polymerizable component (e.g., an ethylenically unsaturated compound, optionally with acid functionality); and water. The refractive index of the combined mixture (measured in the hardened state or the unhardened state) of the polyacid, nanofiller, water and optional polymerizable component is generally within 4 percent of the refractive index of the acid-reactive filler, typically within 3 percent thereof, more typically within 1 percent thereof, and even more typically within 0.5 percent thereof.

U.S. patent application Ser. No. 10/847,805 (Budd et al.) describes dental compositions that can include an acid-reactive nanofiller (i.e., a nanostructured filler) and a hardenable resin (e.g., a polymerizable ethylenically unsaturated compound. The acid-reactive nanofiller can include an oxyfluoride material that is acid-reactive, non-fused, and includes a trivalent metal (e.g., alumina), oxygen, fluorine, an alkaline earth metal, and optionally silicon and/or a heavy metal.

For some embodiments of the present invention that include filler (e.g., dental adhesive compositions), the compositions preferably include at least 1% by weight, more preferably at least 2% by weight, and most preferably at least 5% by weight filler, based on the total weight of the composition. For such embodiments, compositions of the present invention preferably include at most 40% by weight, more preferably at most 20% by weight, and most preferably at most 15% by weight filler, based on the total weight of the composition.

For other embodiments (e.g., where the composition is a dental restorative or an orthodontic adhesive), compositions of the present invention preferably include at least 40% by weight, more preferably at least 45% by weight, and most preferably at least 50% by weight filler, based on the total weight of the composition. For such embodiments, compositions of the present invention preferably include at most 90% by weight, more preferably at most 80% by weight, even more preferably at most 70% by weight filler, and most preferably at most 50% by weight filler, based on the total weight of the composition.

Optional Additives

Optionally, compositions of the present invention may contain solvents (e.g., alcohols (e.g., propanol, ethanol), ketones (e.g., acetone, methyl ethyl ketone), esters (e.g., ethyl acetate), other nonaqueous solvents (e.g., dimethylformamide, dimethylacetamide, dimethylsulfoxide, 1-methyl-2-pyrrolidinone)), and water.

If desired, the compositions of the invention can contain additives such as indicators, dyes, pigments, inhibitors, accelerators, viscosity modifiers, wetting agents, buffering agents, stabilizers, and other similar ingredients that will be apparent to those skilled in the art. Viscosity modifiers include the thermally responsive viscosity modifiers (such as PLURONIC F-127 and F-108 available from BASF Wyandotte Corporation, Parsippany, N.J.) and may optionally include a polymerizable moiety on the modifier or a polymerizable component different than the modifier. Such thermally responsive viscosity modifiers are described in U.S. Pat. No. 6,669,927 (Trom et al.) and U.S. Pat. Publication No. 2004/0151691 (Oxman et al.).

Additionally, medicaments or other therapeutic substances can be optionally added to the dental compositions. Examples include, but are not limited to, fluoride sources, whitening agents, anticaries agents (e.g., xylitol), calcium sources, phosphorus sources, remineralizing agents (e.g., calcium phosphate compounds), enzymes, breath fresheners, anesthetics, clotting agents, acid neutralizers, chemotherapeutic agents, immune response modifiers, thixotropes, polyols, anti-inflammatory agents, antimicrobial agents (in addition to the antimicrobial lipid component), antifungal agents, agents for treating xerostomia, desensitizers, and the like, of the type often used in dental compositions. Combination of any of the above additives may also be employed. The selection and amount of any one such additive can be selected by one of skill in the art to accomplish the desired result without undue experimentation.

Preparation and Use of the Compositions

The dental compositions of the present invention can be prepared by combining an effective amount of an antimicrobial lipid component with a water-dispersible, polymeric film-former component using conventional mixing techniques. The resulting composition may optionally contain a hardenable component, surfactants, fillers, water, solvents, co-solvents, and other additives as described herein.

The compositions of the invention can be supplied in a variety of forms, including one-part systems and multi-part systems. One-part systems or formulations typically include liquids, solutions, dispersions, emulsions, gels, creams, pastes, and the like. Two-part systems include two-part powder/liquid, paste/liquid, and paste/pastesystems. Forms employing multi-part combinations (i.e., combinations of two or more parts), each of which is in the form of a powder, liquid, gel, or paste are also possible. In multi-part systems containing an antimicrobial lipid component, one part typically contains the antimicrobial lipid component and another part contains either the water-dispersible, polymeric film-former component or other components of the final composition. The components of the composition can be included in a kit, where the contents of the composition are packaged to allow for storage of the components until they are needed.

When used as a dental composition, the components of the film-forming compositions can be mixed and clinically applied using conventional techniques. The compositions can be in the form of coatings or sealants that adhere very well to dentin and/or enamel. Optionally, a primer layer can be used on the tooth tissue on which the composition is used. The compositions, e.g., containing a fluoride releasing material, can also provide very good long-term fluoride release.

Typically, the dental compositions include an antimicrobial lipid component, and a water-dispersible, polymeric film-former that is dissolved, dispersed, or emulsified in a solvent, typically a volatile solvent. The solvent can be an alcohol, a ketone, an ester, other non-aqueous solvents, water, or combinations thereof. In use, such compositions can be applied to a tooth surface and the volatile solvent allowed to evaporate to form a film coating (e.g., a film coating layer) on the tooth surface.

The film coating can be continuous (i.e., an integral coating without holes or void spaces) or can be discontinuous (i.e., with holes or void spaces). The film coating can be porous or nonporous with respect to whether moisture vapor and/or oxygen can readily pass through a continuous area of the film coating. The film coating may be adhered to the tooth surface by physical or chemical mechanisms and preferably is generally resistant to wash-off, e.g, from physical wash-off by water, saliva, food, or hot liquids (e.g., tea, coffee, etc.) or by dissolving in such liquids. In some embodiments, it is desirable to have the film coating layer on the tooth surface for a relatively short period of time (e.g., 1-8 hours or less), in which case the film coating layer can be intentionally removed by certain solvents, such as are described herein. In other embodiments, it is desirable to have the film coating layer remain on the tooth surface for relatively long periods of time, e.g. typically greater than 8 hours, preferably greater than 8 days, more preferably greater than 8 weeks, and most preferably greater than 8 months.

In some embodiments of the present invention, the dental composition includes water and a thermally responsive viscosity modifier to form a thermally responsive composition that can be applied, e.g., with a syringe, in a low viscosity state at room temperature to an oral cavity surface (e.g., to a tooth surface). After the composition warms on the oral cavity surface, the composition increases in viscosity to a highly viscous state, such as a gel-like material, that resists run-off from the surface. The thermally responsive composition can optionally contain a hardenable component. Such thermally responsive compositions are described in U.S. Pat. No. 6,669,927 (Trom et al.) and U.S. Pat. Publication No. 2004/0151691 (Oxman et al.). Examples of thermally responsive modifiers include polyoxyalkylene polymers, such as PLURONIC F-127 and PLURONIC F-108 block copolymers of ethylene oxide and propylene oxide available from BASF (Parsippany, N.J.). Such modifiers are typically present in concentrations of about 17% by weight to about 40% by weight (based on the total weight of the composition). The optional hardenable component in thermally responsive compositions is typically an ethylenically unsaturated compound (e.g. a (meth)acrylate compound) that is different from the modifier or an ethylenically unsaturated moiety (e.g. a (meth)acrylate moiety) that is covalently bound to the modifier.

The compositions of the invention are particularly well adapted for use in the form of a wide variety of dental materials, which may be filled or unfilled.

The compositions have utility in a variety of clinical applications where it is desirable to have an antimicrobial composition that can typically be brushed, painted, or sprayed on the tooth surface. Typically, dental applications include compositions for priming, cleansing, lining (e.g., cavity lining), whitening, coloring, protecting (e.g., sealants and coatings to resist microbial damage), remineralization (e.g., by releasing calcium and/or phosphorous ions), and drug delivery. Thus, compositions of the present invention can be used in or as sealants, coatings, varnishes, primers, cavity cleansing agents, whiteners, colorants, desensitizers, cavity liners, remineralizing agents, drug delivery agents, or combinations thereof Such compositions are typically unfilled or lightly filled (e.g., up to 40 wt-% filler, preferably up to 25%, based on the total weight of the composition).

Objects and advantages of this invention are further illustrated by the following examples, but the particular materials and amounts thereof recited in these examples, as well as other conditions and details, should not be construed to unduly limit this invention. Unless otherwise indicated, all parts and percentages are on a weight basis, all water is deionized water, and all molecular weights are weight average molecular weight.

### EXAMPLES

### Test Methods

Turbidity Test Method

A test sample solution (e.g., an isopropanol solution of polymeric film former and antimicrobial component) was uniformly coated to 25-micrometer wet thickness on both sides of 50-micrometer thick corona-treated (under nitrogen) polypropylene. The coatings were allowed to dry in air for 12 hours. Coated sheets were punched to provide 1.5-cm diameter coated discs for antimicrobial testing.

The polymer-coated discs were tested for bacteria attachment and effectiveness according to the following procedure. Overnight culture of Streptococcus mutans (S. mutans) (ATCC#25175) in sterile BHI broth (10⁶ CFU/ml) was prepared. A coated disc was submerged in 9 ml of the bacteria culture for 45 minutes at 25° C. After removing the disc from the culture, the excess culture on the disc was gently rinsed off with de-ionized water and the rinsed disc was placed in a test tube containing 9 ml sterile BHI broth. The turbidity of the culture (with the coated disc) after 24 hours incubation at 37° C. was rated subjectively by the naked eye and defined as follows:

0=a transparent solution (assumed to be free of bacteria growth),

1=a slightly turbid solution (assumed to have minimal bacteria growth),

2=a moderately turbid solution (assumed to have moderate bacteria growth), and

3=a very turbid solution (assumed to have excessive bacteria growth).

The turbidity results were reported as the average of four replications of any given test sample (with an antimicrobial component) and compared to the turbidity result for a control test sample (with no antimicrobial component).

### Abbreviations, Descriptions, and Sources of Materials

| Abbreviation | Description and Source of Material |
|---|---|
| GML-12 | Glycerol monolaurate (Med-Chem Labs, Inc., Galena, IL) |
| PGMC-8 | Propylene glycol monocaprylate (Uniqema, New Castle, |
| | DE) |
| BA | Benzoic acid (Mallinckrodt, St. Louis, MO) |
| DOSS | Dioctyl sodium sulfosuccinate, anionic surfactant |
| | (Cytec Industries, West Paterson, NJ) |
| BHI broth | Brain Heart Infusion broth (VWR, Batavia, IL) |
| AC-315 | AVALURE acrylate-based copolymer |
| | (Noveon, Inc., Cleveland, OH) |
| UR-450 | AVALURE urethane polymer (Noveon, Inc.) |
| AA | Acrylic acid (Sigma-Aldrich) |
| IOA | Isooctyl acrylate (Sigma-Aldrich) |
| IBMA | Isobutyl methacrylate (Sigma-Aldrich) |
| IboA | Isobornyl acrylate (Sigma-Aldrich) |
| DMA-C₁₆Br | Dimethylhexadecylammoniumethyl methacrylate bromide |
| | (Prepared as described for SM-1) |
| DMAEMA | Dimethylaminoethyl methacrylate (Sigma-Aldrich) |

### Starting Material Preparations

Starting Material 1 (SM-1): Synthesis of Dimethylhexadecylammoniumethyl Methacrylate Bromide (DMA-C₁₆Br)

A 500-ml round-bottom flask was charged with 42.2 parts of DMAEMA, 154.7 parts of acetone, 93.2 parts of 1-bromohexadecane (Sigma-Aldrich), and 0.34 parts of BHT. The mixture was stirred for 16 hours at 35° C. and then allowed to cool to room temperature. The resulting white solid precipitate was isolated by filtration, washed with cold ethyl acetate, and dried under vacuum at 40° C. An NMR analysis of the solid product revealed the structure to be pure dimethylhexadecylammoniumethyl methacrylate bromide.

Polymer A: Preparation of Poly(IBMA(60)/AA(20)/DMA-C₁₆Br(20))

IBMA (60 parts), AA (20 parts), DMA-C₁₆Br (20 parts), VAZO-67 (1.0 part), and isopropanol (300 parts) were combined in a reaction vessel and the resulting mixture purged with nitrogen for 2 minutes. The vessel was sealed and maintained at 60° C. in a constant temperature rotating device for 18 hours. The resulting clear viscous polymer solution was utilized in preparing antimicrobial compositions of the present invention. Percent solids analysis revealed a quantitative conversion to polymer that was designated Polymer A and identified as the polymer of IBMA (60 parts), AA (20 parts), and DMA-C₁₆Br (20 parts), with weight ratios indicated in parentheses.

Polymers B—D were prepared as described for Polymer A and are listed as follows with monomeric units and weight ratios indicated:
Polymer B: IBMA (55 parts), AA (20 parts), DMA-C₁₆Br (20 parts), and SiMac (5.0 parts)
Polymer C: IBMA (69 parts), AA (26 parts), and SiMac (5.0 parts)
Polymer D: IBoA (40 parts), IOA (30 parts), and SiMac (30 parts)
   Antimicrobial Component A

Antimicrobial Component A was prepared by combining the ingredients PGMC-8 (1.5 parts), GML-12 (1.5 parts), benzoic acid (1.5 parts) and DOSS (1 part); the resulting mixture was blended in a liquid state at about 60° C. The required quantity of this molten component was subsequently dissolved in a polymer solution to provide antimicrobial compositions used for surface coatings.

### Examples 1-13 and Comparative Examples 1-6

### Antimicrobial Compositions Containing Polymeric Film-Former Components

Antimicrobial compositions containing polymeric film-former components were prepared by dissolving a pre-determined quantity of Antimicrobial Component A into an isopropanol solution containing 20% by weight polymeric film former. The resulting solutions were designated Comparative Examples 1-6 (without Antimicrobial Component A) and Examples 1-13 (with Antimicrobial Component A) and are listed in Table 1.

Examples 1-13 and Comparative Examples 1-6 were evaluated for antimicrobial activity according to the Turbidity Test Method as described herein. The results in terms of Average Turbidity Rating [0 (transparent solution) to 3 (very turbid solution)] are provided in Table 1. A Control Sample (coated disc incubated in BHI broth containing no added bacteria) gave a Turbidity Rating of 0. The greater the Average Turbidity Rating, the greater the turbidity, and therefore the assumption of greater bacteria present in the BHI broth. Thus, a lower Average Turbidity Rating is indicative of greater antibacterial activity (less attached plaque/biofilm) of the coated disc.

TABLE 1
Antimicrobial Adhesive Compositions.
Results of Antimicrobial Evaluations Using Turbidity Test Method

| | | Antimicrobial | Average |
|---|---|---|---|
| | Polymeric Film | Component A | Turbidity |
| Example | Former (20 wt.-%) | (% By Weight) | Rating |
| CE-1 | Polymer A | 0 | 3.0 |
| 1 | Polymer A | 1.0 | 2.0 |
| 2 | Polymer A | 2.5 | 1.0 |
| 3 | Polymer A | 5.0 | 0.75 |
| CE-2 | Polymer B | 0 | 3.0 |
| 4 | Polymer B | 1.0 | 2.0 |
| 5 | Polymer B | 2.5 | 1.5 |
| 6 | Polymer B | 5.0 | 1.25 |
| CE-3 | Polymer C | 0 | 3.0 |
| 7 | Polymer C | 1.0 | 1.75 |
| 8 | Polymer C | 2.5 | 2.0 |
| 9 | Polymer C | 5.0 | 2.0 |
| CE-4 | Polymer D | 0 | 2.75 |
| 10 | Polymer D | 1.0 | 1.5 |
| 11 | Polymer D | 2.5 | 2.0 |
| CE-5 | AC-315 | 0 | 3.0 |
| 12 | AC-315 | 5.0 | 1.75 |
| CE-6 | UR-450 | 0 | 3.0 |
| 13 | UR-450 | 5.0 | 2.0 |

The results from Table 1 show that all of the inventive antimicrobial compositions (Examples 1-13) imparted at least some degree of antimicrobial activity to the coated discs as compared to the corresponding coated discs with no added antimicrobial component (Comparative Examples 1-6), and that generally the imparted antimicrobial activity was dose dependent.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

What is claimed is:

1. A dental composition comprising:
an effective amount of an antimicrobial lipid component comprising a (C7-C12)saturated fatty acid ester of a polyhydric alcohol, a (C8-C22)unsaturated fatty acid ester of a polyhydric alcohol, a (C7-C12)saturated fatty ether of a polyhydric alcohol, a (C8-C22)unsaturated fatty ether of a polyhydric alcohol, an alkoxylated derivative thereof, or combinations thereof, wherein the alkoxylated derivative has less than 5 moles of alkoxide per mole of polyhydric alcohol; with the proviso that for polyhydric alcohols other than sucrose, the esters comprise monoesters and the ethers comprise monoethers, and for sucrose the esters comprise monoesters, diesters, or combinations thereof, and the ethers comprise monoethers, diethers, or combinations thereof; and
a water-dispersible, polymeric film-former.

2. The dental composition of claim 1 further comprising an effective amount of an enhancer component distinct from the antimicrobial lipid component.

3. The dental composition of claim 2 wherein the enhancer component comprises a carboxylic acid.

4. The dental composition of claim 2 wherein the enhancer component comprises an alpha-hydroxy acid.

5. The dental composition of claim 2 wherein the enhancer component comprises an alpha-hydroxy acid, a beta-hydroxy acid, a chelating agent, a (C1-C4)alkyl carboxylic acid, a (C6-C12)aryl carboxylic acid, a (C6-C12)aralkyl carboxylic acid, a (C6-C12)alkaryl carboxylic acid, a phenolic compound, a (C1-C10)alkyl alcohol, an ether glycol, or combinations thereof.

6. The dental composition of claim 2 wherein the total concentration of the enhancer component relative to the total concentration of lipid component is within a range of 10:1 to 1:300, on a weight basis.

7. The dental composition of claim 1 further comprising an effective amount of a surfactant component distinct from the antimicrobial lipid component.

8. The dental composition of claim 7 wherein the surfactant component comprises a sulfonate surfactant, a sulfate surfactant, a phosphonate surfactant, a phosphate surfactant, a poloxamer surfactant, a cationic surfactant, or mixtures thereof.

9. The dental composition of claim 8 wherein the surfactant component comprises a sulfonate surfactant, a sulfate surfactant, a poloxamer surfactant, or mixtures thereof.

10. The dental composition of claim 9 wherein the surfactant component is dioctyl sodium sulfosuccinate.

11. The dental composition of claim 9 wherein the surfactant component is a poloxamer comprising a copolymer of polyethylene oxide and polypropylene oxide.

12. The dental composition of claim 7 wherein the total concentration of the surfactant component to the total concentration of antimicrobial lipid component is within a range of 5:1 to 1:100, on a weight basis.

13. The dental composition of claim 1 further comprising a hardenable component.

14. The dental composition of claim 13 wherein the hardenable component comprises an ethylenically unsaturated compound.

15. The dental composition of claim 14 wherein the ethylenically unsaturated compound is a (meth)acrylate compound.

16. The dental composition of claim 14 wherein the ethylenically unsaturated compound is selected from the group consisting of an ethylenically unsaturated compound with acid functionality, an ethylenically unsaturated compound without acid functionality, and combinations thereof.

17. The dental composition of claim 13 further comprising an initiator system.

18. The dental composition of claim 1 wherein the antimicrobial lipid component comprises glycerol monolaurate, glycerol monocaprate, glycerol monocaprylate, propylene glycol monolaurate, propylene glycol monocaprate, propylene glycol monocaprylate, or combinations thereof.

19. The dental composition of claim 1 wherein the antimicrobial lipid component is present in an amount of at least 0.1 wt-%.

20. The dental composition of claim 19 wherein the antimicrobial lipid component comprises a monoester of a polyhydric alcohol, a monoether of a polyhydric alcohol, or an alkoxylated derivative thereof, and the antimicrobial lipid component further includes no greater than 15 wt-%, based on the total weight of the antimicrobial lipid component, of a di- or tri-ester, a di- or tri-ether, alkoxylated derivative thereof, or combinations thereof.

21. The dental composition of claim 1 further comprising a filler.

22. The dental composition of claim 1 wherein the water-dispersible, polymeric film-former comprises a repeating unit that includes a polar or polarizable group.

23. The dental composition of claim 22 wherein the polar or polarizable group is derived from vinylic monomers.

24. The dental composition of claim 22 wherein the water-dispersible polymeric film-former further comprises a repeating unit that includes a fluoride releasing group, a repeating unit that includes a hydrophobic hydrocarbon group, a repeating unit that includes a graft polysiloxane chain, a repeating unit that includes a hydrophobic fluorine-containing group, a repeating unit that includes a modulating group, or combinations thereof.

25. The dental composition of claim 1 wherein the water-dispersible polymeric film-former includes reactive groups.

26. The dental composition of claim 25 wherein the reactive groups are selected from the group consisting of ethylenically unsaturated groups, epoxy groups, silane moieties capable of undergoing a condensation reaction, and combinations thereof.

27. The dental composition of claim 1 wherein the water-dispersible polymeric film-former includes a polymer having repeating amide functional groups.

28. The dental composition of claim 1 wherein the water-dispersible polymeric film-former includes a polymer having repeating acrylate functional groups.

29. The dental composition of claim 1 wherein the water-dispersible polymeric film-former includes a polymer having repeating N-isopropylamide functional groups.

30. The dental composition of claim 1 wherein the water-dispersible polymeric film-former includes a polymer having repeating urethane functional groups.

31. The dental composition of claim 1 wherein the composition further comprises a solvent.

32. The dental composition of claim 31 wherein the solvent is volatile.

33. The dental composition of claim 1 wherein the composition is selected from the group consisting of coatings, varnishes, sealants, primers, whiteners, cavity cleansing agents, desensitizers, cavity liners, remineralizing agents, drug delivery agents, colorants, and combinations thereof.

34. A method of forming a polymeric film coating layer on an oral cavity surface, the method comprising:
combining an antimicrobial lipid component and a water-dispersible, polymeric film-former to form the dental composition of claim 1;
applying the composition to the oral cavity surface; and
allowing the film coating to form on the oral cavity surface.

35. A method of forming a polymeric film coating layer on an oral cavity surface, the method comprising:
combining an antimicrobial lipid component, a water-dispersible, polymeric film-former, and a volatile solvent to form the dental composition of claim 32;
applying the composition to the oral cavity surface; and
allowing the evaporation of at least a portion of the volatile solvent to form the film coating layer on the oral cavity surface.
